SEQUENCE LISTING (14 sequences)

SEQ ID NO: 1 - protein, 568 aa, Paramecium bursaria Chlorella virus 1
MGKNIIIMVSWYTIITSNLIAVGGASLILAPAITGYVLHWNIALSTIWGVSAYGIFVFGFFLAQVLFSELNRKRLRKWISLRPKGWNDVRLAVIIAGYREDPYMFQKCLESVRDSDYGNVARLICVIDGDEDDDMRMAAVYKAIYNDNIKKPEFVLCESDDKEGERIDSDFSRDICVLQPHRGKRECLYTGFQLAKMDPSVNAVVLIDSDTVLEKDAILEVVYPLACDPEIQAVAGECKIWNTDTLLSLLVAWRYYSAFCVERSAQSFFRTVQCVGGPLGAYKIDIIKEIKDPWISQRFLGQKCTYGDDRRLTNEILMRGKKVVFTPFAVGWSDSPTNVFRYIVQQTRWSKSWCREIWYTLFAAWKHGLSGIWLAFECLYQITYFFLVIYLFSRLAVEADPRAQTATVIVSTTVALIKCGYFSFRAKDIRAFYFVLYTFVYFFCMIPARITAMMTLWDIGWGTRGGNEKPSVGTRVALWAKQYLIAYMWWAAVVGAGVYSIVHNWMFDWNSLSYRFALVGICSYIVFIVIVLVVYFTGKITTWNFTKLQKELIEDRVLYDATTNAQSV

SEQ ID NO: 2 - protein, 588 aa, Xenopus laevis
MKEKAAETMEIPEGIPKDLEPKHPTLWRIIYYSFGVVLLATITAAYVAEFQVLKHEAILFSLGLYGLAMLLHLMMQSLFAFLEIRRVNKSELPCSFKKTVALTIAGYQENPEYLIKCLESCKYVKYPKDKLKIILVIDGNTEDDAYMMEMFKDVFHGEDVGTYVWKGNYHTVKKPEETNKGSCPEVSKPLNEDEGINMVEELVRNKRCVCIMQQWGGKREVMYTAFQAIGTSVDYVQVCDSDTKLDELATVEMVKVLESNDMYGAVGGDVRILNPYDSFISFMSSLRYWMAFNVERACQSYFDCVSCISGPLGMYRNNILQVFLEAWYRQKFLGTYCTLGDDRHLTNRVLSMGYRTKYTHKSRAFSETPSLYLRWLNQQTRWTKSYFREWLYNAQWWHKHHIWMTYESVVSFIFPFFITATVIRLIYAGTIWNVVWLLLCIQIMSLFKSIYACWLRGNFIMLLMSLYSMLYMTGLLPSKYFALLTLNKTGWGTSGRKKIVGNYMPILPLSIWAAVLCGGVGYSIYMDCQNDWSTPEKQKEMYHLLYGCVGYVMYWVIMAVMYWVWVKRCCRKRSQTVTLVHDIPDMCV

SEQ ID NO: 3 - protein, 578 aa, Homo sapiens
MRQQDAPKPTPAACRCSGLARRVLTIAFALLILGLMTWAYAAGVPLASDRYGLLAFGLYGAFLSAHLVAQSLFAYLEHRRVAAAARGPLDAATARSVALTISAYQEDPAYLRQCLASARALLYPRARLRVLMVVDGNRAEDLYMVDMFREVFADEDPATYVWDGNYHQPWEPAAAGAVGAGAYREVEAEDPGRLAVEALVRTRRCVCVAQRWGGKREVMYTAFKALGDSVDYVQVCDSDTRLDPMALLELVRVLDEDPRVGAVGGDVRILNPLDSWVSFLSSLRYWVAFNVERACQSYFHCVSCISGPLGLYRNNLLQQFLEAWYNQKFLGTHCTFGDDRHLTNRMLSMGYATKYTSRSRCYSETPSSFLRWLSQQTRWSKSYFREWLYNALWWHRHHAWMTYEAVVSGLFPFFVAATVLRLFYAGRPWALLWVLLCVQGVALAKAAFAAWLRGCLRMVLLSLYAPLYMCGLLPAKFLALVTMNQSGWGTSGRRKLAANYVPLLPLALWALLLLGGLVRSVAHEARADWSGPSRAAEAYHLAAGAGAYVGYWVAMLTLYWVGVRRLCRRRTGGYRVQV

SEQ ID NO: 4 - protein, 551 aa, Homo sapiens
MHCERFLCILRIIGTTLFGVSLLLGITAAYIVGYQFIQTDNYYFSFGLYGAFLASHLIIQSLFAFLEHRKMKKSLETPIKLNKTVALCIAAYQEDPDYLRKCLQSVKRLTYPGIKVVMVIDGNSEDDLYMMDIFSEVMGRDKSATYIWKNNFHEKGPGETDESHKESSQHVTQLVLSNKSICIMQKWGGKREVMYTAFRALGRSVDYVQVCDSDTMLDPASSVEMVKVLEEDPMVGGVGGDVQILNKYDSWISFLSSVRYWMAFNIERACQSYFGCVQCISGPLGMYRNSLLHEFVEDWYNQEFMGNQCSFGDDRHLTNRVLSLGYATKYTARSKCLTETPTEYLRWLNQQTRWSKSYFREWLYNSLWFHKHHLWMTYEAVITGFFPFFLIATVIQLFYRGRIWNILLFLLTVQLVGLIKSSFASALRGNIVMVFMSFYSVLYMSSLLPAKMFAIATINKAGWGTSGRKTIVVNFIGLIPITVWFTILLGGVCYTIWRETKKPFSESEKIVLAVGAILYACYWVMLLTMYVSLVMKCGRRRKEPQHDLVLA

SEQ ID NO: 5 - protein, 551 aa, Xenopus laevis
MHCERFICILRIIGTTLFGVSLLLGISAAYIVGYQFIQTDNYYFSFGLYGAILALHLIIQSLFAFLEHRKMKRSLETPIKLNKSVALCIAAYQEDEDYLRKCLLSVKRLTYPGMKVIMVIDGNSDDDLYMMNIFREIMGNDSCATYVWKNNFHMKGPNETDETHRESMQHVTQMVLSNRNVCIMQKWNGKREVMYTAFKALGRSVDYVQVCDSDTVLDPASSVEMVKVLEEDIMVGGVGGDVQILNKYDSWISFLSSVRYWMAFNIERACQSYFGCVQCISGPLGMYRNSLLHEFIEDWYNQEFLGSQCSFGDDRHLTNRVLSLGYATKYTARSKCLTETPTEYLRWLNQQTRWSKSYFREWLYNSLWFHKHHLWMTYEAVITGFFPFFLIATVIQLFYRGRIWNILLFLLTVQLVGLIKSSFASALRGNIVMVFMSFYSVLYMSSLLPAKMFAIATINKAGWGTSGRKTIVVNFIGLIPITVWFTILLGGVCYTIWRETKKPFSESEKIVLAVGAILYACYWVMLLTMYVSLVMKCGRRRKEPQHDLVLA

SEQ ID NO: 6 - protein, 553 aa, Homo sapiens
MPVQLTTALRVVGTSLFALAVLGGILAAYVTGYQFIHTEKHYLSFGLYGAILGLHLLIQSLFAFLEHRRMRRAGQALKLPSPRRGSVALCIAAYQEDPDYLRKCLRSAQRISFPDLKVVMVVDGNRQEDAYMLDIFHEVLGGTEQAGFFVWRSNFHEAGEGETEASLQEGMDRVRDVVRASTFSCIMQKWGGKREVMYTAFKALGDSVDYIQVCDSDTVLDPACTIEMLRVLEEDPQVGGVGGDVQILNKYDSWISFLSSVRYWMAFNVERACQSYFGCVQCISGPLGMYRNSLLQQFLEDWYHQKFLGSKCSFGDDRHLTNRVLSLGYRTKYTARSKCLTETPTKYLRWLNQQTRWSKSYFREWLYNSLWFHKHHLWMTYESVVTGFFPFFLIATVIQLFYRGRIWNILLFLLTVQLVGIIKATYACFLRGNAEMIFMSLYSLLYMSSLLPAKIFAIATINKSGWGTSGRKTIVVNFIGLIPVSIWVAVLLGGLAYTAYCQDLFSETELAFLVSGAILYGCYWVALLMLYLAIIARRCGKKPEQYSLAFAEV

SEQ ID NO: 7 - protein, 574 aa, Xenopus laevis
MPEDSLTVEAVQSRAQGGSSMAEQEAANKMPGKFQTGLRVLGTSLFALLVLGGILAAYVTGYQFIHTDRHHLSFGLYGAILGLHLLSQSLFAFLEHRKMRGGGRRPTGRSTVALCIAAYQEDPEYLRKCLRSVCRLSYPHLRVIMVVDGNTEEDRYMMDIFREVMGSEGTCCYIWDKNYHESEEGGQEGERGVQEMVKNFQYVCIMQKWGGKREVMYTAFRALGNSVDYVQVCDSDTVLDPACTAEMLRILEEDPKVGGVGGDVQILNKYESWISFLSSVRYWMAFNVERACQSYFGCVQCISGPLGMYRNSLLQYFLEDWYHQTFLGQKCSFGDDRHLTNRVLSMGFRTKYTARSRCLTETPTRYLRWLNQQTRWSKSYFREWLYNALWFHKHHLWMTYESVVTGFFPFFLVATVVQLFYRGRVWNILLFLLTVQLVGIVKATYACFLRGNAEMIFMSLYSLLYMTSLLPAKIFAVMTINKSGWGTSGRRKLVVNFMGMVPVSVWFCILLGGLVYTAYCQSQDPFTETELLFLVTGAILYGCYWVALLSLYLALIARRCGKRQELYNLALAEV

SEQ ID NO: 8 - DNA, 4348 bp, Xenopus laevis
  source 1..4348: mol_type = genomic DNA
gaattcaaaaataagctcctgctttgaggccactggggagcaacatccaaggggttggggagcaacatgttactcacgagctactggttggggatcactgacctaaggtattcaccccaaggtccgaccaaagactcattgggcctagctttgccaacaaggggtaattgtggttgttccatagtgggattatgaaacatttcaacccaaaatcttctcctgtgttaaatttgataaaaatatcggtaaaaaaaaaagcccccagagtatcacaatttttatttcctattacatttatttgttacttttcaaattaaaaataagaggttccaggtcagggctgcaccaatcaccaagcaatgctccctcccctcatttgctatggagtggcattccagcctattgttatgcaggggtgtggcctatacaggagcctgcaggtatataaacccaggtcagagtggtcattgtcactaaaaacttggcatatcccatgtgtgtgtcttggggcctcttcattcggcaacatgaaggaaaaagctgcaggtaagtgtctttttctgaacctttttataattcctttattcacccccccccaacgcataggattttaggagacccacattttaaagggaaaatacaccccaaatcttttttatgcttaatgaatgaatttaaagttatatctaagtgtaattgcactagaatgctgcattggtattggaaacttgcctataattaattttattggggtggagttccccattaaattgtgccttgttactattctcttatttgactgttcttacacataatttcatattcactaaaacataatccacgttattataaggcaaatttataaatgccaaattgtttctcttagtcagtacagtataagggtatagcttattgtgtgcccagaacattccttctctgtatatttgtatttatacatatgggaggaggaggtgccatattgattcccttagacagtacagtataagggtatagcttattgtgtgcccagaacattccttctctgtatatttgtatttatacatatgggaggaggtgccatattgattcccttagacagtacagtatgagggtatagcttattgtgtgcccagaacattccttctctgtatatttgtatttatacatatgggaggaggtgccatattgattcccttacacagtacagtatgagggtatagcttattgtgtgcccagaacattccttctctgtatatttgtatttatacatatgggaggaggaggtgccatattgattccctagacagtacagtatgagggtatagtttattgtgtgcccggaacattctttctctgtatatttgtatttatacatatgggaggaggaggtgccctattgattcccttagaccatacagtatgagggaacagcttattgtgtgcccagagcattcctactctgtatatgtaacagtttatccataatttctaatccttctctttttctttccttagaaacaatggagattcctgaagggatccccaaagatctagagccaaaacaccccaccctttggaggataatttattattcttttggtgtggtgctattagctaccattacagcagcctatgtggcagagttccaggtcctcaaacatgaagccattctcttctcccttgggctttatggtcttgcaatgcttctccacctgatgatgcagagcctctttgccttcctggagatacgcagggtaaataagagtgagcttccttgcagctttaagaagacagtggctctgaccattgctgggtatcaggagaaccctgagtacctgataaagtgcttggaatcctgcaagtatgtgaaataccccaaagataaactcaagatcattttggtcatcgatgggaacacagaggatgatgcctacatgatggagatgttcaaagacgtgttccacggtgaagatgtaggcacctacgtatggaagggaaattaccacactgttaaaaagcctgaggagaccaataagggatcctgtcctgaggtttctaagcccttgaatgaagatgaaggtatcaatatggtggaagagcttgttagaaacaagagatgtgtgtgcatcatgcaacagtggggcggaaaaagagaggtcatgtacacagcattccaggccattgggacttctgtggactatgtacaggtgggtctatttatctttcaaaaatggccatggtagagtctgattcccttatttatgttctcagcatgttctcctccgatgttgtgcttcttccttttatttgcctctttggatcttcttgtccattcatgttatgttgttattcatgtgtttcatcttttaattccattaattataggtctgtgactcggacaccaaactggatgaactggcaacagtggaaatggtgaaggttctggagtccaatgacatgtacggcgcagtgggaggagacgttcgcattctgaacccttatgattccttcattagtttcatgagcagcctgcgttactggatggcgtttaacgtggagagggcctgccagtcttacttcgactgcgtgtcctgtataagtggacctctgggtaggtggcccaaaaatattatataactttggttccatgtaatggatcttgttgccatgtgtatcataatcgcaagctcatcattacatttatgttcccctatttgaaggaatgtaccggaacaacattctccaggtgtttttggaagcctggtacagacagaaatttttgggaacctattgtactttgggagatgatagacatctgacaaaccgagtgctcagcatgggatatcgcaccaagtaagttgtctgtgaatgtggtgagcaattctatgcggggaaagcagtgggacagattgtgacttttcatctgatgatctctctacgtctttctctattactcaacatgaccaataattgacttatttgtccctttctctctttaagatacacccacaaatccagagcattctccgaaactccatccctgtatctccggtggttgaaccagcaaacccggtggaccaagtcctacttccgagagtggctgtataatgcccagtggtggcacaagcatcacatctggatgacctatgagtctgtggtgtccttcatctttcccttcttcatcactgccactgttatccgcctcatctatgccggcaccatatggaatgttgtgtggctcctcctgtgcatccagatcatgtctctcttcaaatccatctatgcctgctggctccgcggcaacttcattatgctcctgatgtctctctactccatgctgtacatgactgggcttctgccatccaagtactttgccttgttgaccttaaacaagaccggttggggaacatctgggcgcaagaagatagtaggcaactacatgccaatactgcccctgtccatatgggcagctgttctgtgtggaggggtgggttatagtatctatatggactgtcaaaatgactggagcacccctgaaaagcaaaaggagatgtaccatctattgtatgggtgtgtgggctatgtaatgtattgggtaatcatggctgtgatgtactgggtctgggtgaagaggtgctgcaggaagaggtcccaaactgtcaccctggtccatgacattcctgatatgtgtgtttaaaggaatactgctatcacacgatctatagtccctttctattattgcaacctatgtgtattttactgtcaataggtgtcatcatcatctatggctacctgctccataacaaaaccatgaaatctatagccagcaacaagaagcggcgactattattttcattctcatattgagagacggtttttgtctcctggtttttatccagtctccaatatatcacaaaaaaggttttcaagttattaataatgaaattgctattaaaagcagcttctgtcctttgcgatatctttctccgtttgacagaaacaatgtagcaataatgagacccttaatactccagttcccacaatgccttgttcacttggtaacccccttcttctcaggtatgttaatcaccaaacatgcaaggtatcatgggaattaggtccttgcttgaggaggttggactgctaatacaatgtagcaattgcatttacatttaactttaatatcactgacaaaattctatgtatattggggaaaattgcctttaagacagtttatattgctcaagtgtttttatttacattttttattcctacaaatatatcataaatctctccctaatcccctgttgctgtgatgttaataaagttatgtttccttgtactgtaa

SEQ ID NO: 9 - protein, 588 aa, Xenopus laevis; identical to SEQ ID NO: 2

SEQ ID NO: 10 - RNA, 2950 nt, Xenopus laevis
  source 1..2950: mol_type = genomic RNA
gaattcaaaaataagctcctgctttgaggccactggggagcaacatccaaggggttggggagcaacatgttactcacgagctactggttggggatcactgacctaaggtattcaccccaaggtccgaccaaagactcattgggcctagctttgccaacaaggggtaattgtggttgttccatagtgggattatgaaacatttcaacccaaaatcttctcctgtgttaaatttgataaaaatatcggtaaaaaaaaagcccccagagtatcacaatttttatttcctattacatttatttgttacttttcaaattaaaaataagaggttccaggtcagggctgcaccaatcaccaagcaatgctccctcccctcatttgctatggagtggcattccagcctattgttatgcaggggtgtggcctatacaggagcctgcaggtatataaacccaggtcagagtggtcattgtcactaaaaacttggcatatcccatgtgtgtgtcttggggcctcttcattcggcaacatgaaggaaaaagctgcagaaacaatggagattcctgaagggatccccaaagatctagagccaaaacaccccaccctttggaggataatttattattcttttggtgtggtgctattagctaccattacagcagcctatgtggcagagttccaggtcctcaaacatgaagccattctcttctcccttgggctttatggtcttgcaatgcttctccacctgatgatgcagagcctctttgccttcctggagatacgcagggtaaataagagtgagcttccttgcagctttaagaagacagtggctctgaccattgctgggtatcaggagaaccctgagtacctgataaagtgcttggaatcctgcaagtatgtgaaataccccaaagataaactcaagatcattttggtcatcgatgggaacacagaggatgatgcctacatgatggagatgttcaaagacgtgttccacggtgaagatgtaggcacctacgtatggaagggaaattaccacactgttaaaaagcctgaggagaccaataagggatcctgtcctgaggtttctaagcccttgaatgaagatgaaggtatcaatatggtggaagaacttgttagaaacaagagatgtgtgtgcatcatgcaacagtggggcggaaaaagagaggtcatgtacacagcattccaggccattgggacttctgtggactatgtacaggtctgtgactcggacaccaaactggatgaactggcaacagtggaaatggtgaaggttctggaatccaatgacatgtacggcgcagtgggaggagacgttcgcattctgaacccttatgattccttcattagtttcatgagcagcctgcgttactggatggcgtttaacgtggagagggcctgccagtcttacttcgactgcgtgtcctgtataagtggacctctgggaatgtaccggaacaacattctccaggtgtttttggaagcctggtacagacagaaatttttgggaacctattgtactttgggagatgatagacatctgacaaaccgagtgctcagcatgggatatcgcaccaaatacacccacaaatccagagcattctccgaaactccatccctgtatctccggtggttgaaccagcaaacccggtggaccaagtcctacttccgagagtggctgtataatgcccagtggtggcacaagcatcacatctggatgacctatgagtctgtggtgtccttcatctttcccttcttcatcactgccactgttatccgcctcatctatgccggcaccatatggaatgttgtgtggctcctcctgtgcatccagatcatgtctctcttcaaatccatctatgcctgctggctccgcggcaacttcattatgctcctgatgtctctctactccatgctgtacatgactgggcttctgccatccaagtactttgccttgttgaccttaaacaagaccggttggggaacatctgggcgcaagaagatagtaggcaactacatgccaatactgcccctgtccatatgggcagctgttctgtgtggaggggtgggttatagtatctatatggactgtcaaaatgactggagcacccctgaaaagcaaaaggagatgtaccatctattgtatgggtgtgtgggctatgtaatgtactgggtaatcatggctgtgatgtactgggtctgggtgaagaggtgctgcaggaagaggtcccaaactgtcaccctggtccatgacattcctgatatgtgtgtttaaaggaatactgctatcacacgatctatagtcccttctattgttgcaacctatgtgtattttactgttcaataggtgtcatcatcatctatggctacctactccataacaaaaccatagaatctatagccagcaacaagaagcggcgactattattttcattctcatattgagagacggtttttgtctcctggtttttatccagtctccaatatatcacaaaaaaggttttcaagttattaataatgaaattgctattaaaagcagcttctgtctgttctttgcgatatctttctccgtttgacagaaacaatgtagcaataatgagacccttaatactctagttcccacaatgccttgttcacttggtaatccccttcttctcaggtatgttaatcaccaaacatgcaaggtatcatgggaattaggtccttgcttgaggaggttggactgctaatacaatgcagcaattgcatttacatttaactttaatatcactgacaaaattctatgtatattggggaaaattgcctttaagacagtttatattgctcaagtgtttttatttacattttttattcctacaaatatatcataaatctctccctaatcccctgttgctgtgatgttaataaagttatgtttccttgtactgtaaaa

SEQ ID NO: 11 - protein, 8 aa, Xenopus laevis
WGTSGRKR

SEQ ID NO: 12 - protein, 5 aa, Paramecium bursaria Chlorella virus 1
WGTRG

SEQ ID NO: 13 - protein, 4 aa, Xenopus laevis
GLYG

SEQ ID NO: 14 - protein, 4 aa, Xenopus laevis
GDDR

(19) United States
(12) Patent Application Publication
(10) Pub. No.: US 2026/0286413 A1
(43) Pub. Date: Sep. 24, 2026

(54) HYALURONAN SYNTHESIS, SECRETION AND LENGTH CONTROL

(71) Applicants: UNIVERSITY OF VIRGINIA, Charlottesville, VA (US); University of Virginia Patent Foundation, Charlottesville, VA (US)

(72) Inventors: Jochen Zimmer, Charlottesville, VA (US); Ireneusz Górniak, Charlottesville, VA (US)

(21) Appl. No.: 19/480,272

(22) PCT Filed: May 2, 2024
(86) PCT No.: PCT/US2024/027518

§ 371 Date: Oct. 30, 2025

Related U.S. Application Data
(60) Provisional application No. 63/499,559, filed on May 2, 2023.

(51) Int. Cl. C12P 19/26; C12N 9/10; C12P 19/18

(52) U.S. Cl. CPC C12P 19/26; C12N 9/1051; C12P 19/18; C12Y 204/01212

(57) ABSTRACT

Provided herein are compositions and methods of hyaluronan biosynthesis, secretion and length control.

### PRIORITY

This application claims the benefit of priority of U.S. provisional patent application No. 63/499,559, filed on May 2, 2023, the benefit of priority of which is claimed hereby, and which is incorporated by reference herein in its entirety.

### GOVERNMENT GRANT SUPPORT

This invention was made with government support under GM144130 awarded by the National Institutes of Health. The government has certain rights in the invention.

### BACKGROUND OF THE INVENTION

Hyaluronan (HA) is a component of the vertebrate extracellular matrix. It is a heteropolysaccharide of alternating N-acetylglucosamine (GlcNAc) and glucuronic acid (GlcA) units reaching several megadaltons in healthy tissues.

### SUMMARY

HA is synthesized and secreted in a coupled reaction by HA-synthase (HAS). Structural snapshots of HAS provide insights into HA biosynthesis, from substrate recognition to HA elongation and translocation. The coordination of the UDP product by a conserved gating loop is revealed, the extension of a GlcNAc primer with GlcA is monitored, and the opening of a secretion channel that coordinates a translocating HA polymer is captured. Further, channel-lining residues that modulate HA product lengths were identified. Integrating structural and biochemical analyses provides a mechanism for polysaccharide length control based on enzymatic activity and HA coordination.

One embodiment provides a method to synthesize Hyaluronan (HA) product and modulate HA's length, alter catalytic rate of HA production and/or alter polydispersity of HA comprising expressing a mutant nucleic acid sequence coding for mutant HA synthase (HAS) in a host cell, wherein the mutant HA synthase has one or more substitutions in WGTSGRK/R motif or WGTRG motif. In one embodiment, the catalytic rate of the mutant synthase is increased or reduced as compared to a wild type synthase. In another embodiment, the length of HA is shorter or longer compared to HA synthesized with a wild type synthase. In one embodiment, the polydispersity the HA is greater or less than the polydispersity of HA synthesized with a wild type synthase. In one embodiment, Trp of the WGTSGRK/R motif is substituted. In one embodiment, the Trp is substituted with Ala or Phe. In one embodiment, Thr of the WGTSGRK/R motif is substituted. In one embodiment, Thr is substituted with Ala or Ser. In one embodiment, Arg of the WGTSGRK/R motif is substituted. In one embodiment, Arg is substituted with Ala or Lys. In one embodiment, at least one Gly in WGTRG is substituted. In one embodiment, at least one Gly is substituted with Ala. In one embodiment, Arg in WGTRG is substituted. In one embodiment, Arg is substituted with Lys.

One embodiment provides a method to synthesize Hyaluronan (HA) product and modulate HA's length, alter catalytic rate of HA production and/or alter polydispersity of HA comprising expressing a mutant nucleic acid sequence coding for mutant HA synthase (HAS) in a host cell, wherein the mutant HA synthase has one or more substitutions in a TM channel. In one embodiment, the one or more substitutions in the TM channel are at Arg287, Arg296, H72, and/or K448. In one embodiment, the one or more substitutions are Arg287Ala, Arg287Lys, Arg296Ala, Arg296Lys, H72A, K448A, K448R, and/or H72F.

One embodiment provides a method to synthesize Hyaluronan (HA) product and modulate HA's length, alter catalytic rate of HA production and/or alter polydispersity of HA comprising expressing a mutant nucleic acid sequence coding for mutant HA synthase (HAS) in a host cell, wherein the mutant HA synthase has one or more substitutions in the active site channel, wherein the substitutions are at C307 or C337. In one embodiment, C307 and/or C337 are substituted independently with Ala or Ser.

In one embodiment, the HA synthesis reaction is carried out for about 30 minutes to about 10 hours, such as about 30 min, about 60 min, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours or about 10 hours.

In one embodiment, the HA synthesized in smaller or larger than wild type, such as about 1.6 or greater MDa.

### BRIEF DESCRIPTION OF THE DRAWINGS

FIGS. 1A-1H. In vitro hyaluronan biosynthesis. a, HAS couples HA synthesis with membrane translocation. b, HA disaccharide structure. c, S200 size exclusion chromatogram of purified X1HAS1. Inset: Coomassie-stained SDS-PAGE. d, Time-course of in vitro HA biosynthesis monitored by agarose gel electrophoresis and Stains-all staining. H*: Hyaluronidase digestion. The molecular weight marker represents HA size standards. e, Comparison of HA synthesized in vitro by Streptococcus equisimilis (Se), Chlorella virus (Cv) and Xenopus laevis (X1) HAS1. All proteins were assayed after purification in the GDN detergent. f, Catalytic activity depending on substrate concentration. One substrate concentration was at 2.5 mM while the other was varied as indicated. Activity is measured by quantifying the released UDP in an enzyme coupled reaction. g, The same as in panel (f) but by monitoring HA formation. h, Increasing HA molecular weight in the absence of product inhibition. A standard synthesis reaction was supplemented three times with 2.5 mM UDP-GlcA and the indicated amounts of UDP-GlcNAc while enzymatically converting UDP to UTP. Arrowheads in panels g and h indicate the 1650 kDa HA marker for orientation.

FIGS. 2A-2E. Structure of Xenopus laevis HAS1. a, Representative 2D class averages and electron potential map of X1HAS1. The map was contoured at σ=9 r.m.s.d and is overlaid with an ab initio volume to visualize the detergent micelle. b, Domain organization of X1HAS1 and Fab binding site. c, Architecture of X1HAS1. The catalytic domain, amphipathic interface helices (IFH), and transmembrane regions are colored light blue, hot pink, and pink, respectively. d, Surface representation of X1HAS1 indicating a curved channel with lateral pathway. e, Cartoon representation of X1HAS1 highlighting the positions of the N-terminal extension and the unresolved gating loop above the active site (dashed line).

FIGS. 3A-3C. A HA-HAS translocation intermediate. a, Left panel: X1HAS1 structure with a nascent HA nonasaccharide. Right panels: Stick representations of residues coordinating HA inside X1HAS1 TM channel colored by the electrostatic surface potential calculated in PyMOL using the APBS plugin 49,51 (red to blue, −10 to 10 kT). The nascent HA polymer is shown as ball-and-sticks with carbons colored yellow and orange for GlcA and GlcNAc units, respectively. b, Gating transitions upon HA formation. Shown is a superimposition of the TM regions only in the presence (pink) and absence (white) of a translocating HA polymer. c, Gating loop insertion into the catalytic pocket. Conserved residues contacting UDP are shown as sticks. UDP is shown as ball-and-sticks colored cyan for carbon atoms.

FIGS. 4A-4G. Modulation of HA length. a, X1HAS1 composite model showing mutagenized residues forming the active site (colored light blue and deep teal for residues of the GT domain and gating loop, respectively) or the HA channel (colored pink and hot pink for residues of TMHs and IFHs, respectively). b, Mutagenesis of active-site-lining residues. In vitro HA biosynthesis by the indicated X1HAS1 mutants for 1 h or 8 h (short and long reactions, respectively). WT: Wild-type, H*—hyaluronidase-treated samples. Agarose gels were stained with Stains-all. The dashed line roughly indicates the WT's highest molecular weight product. c, Catalytic rates of the mutants shown in (b). Rates, normalized to WT, report substrate turnovers and were determined by quantifying UDP release in real time during HA synthesis. d, Catalytic activity of gating loop mutants of CvHAS. Activity was determined by quantifying 3H-labeled HA by scintillation counting and is expressed relative to WT 10. Numbers in parenthesis indicate residue numbers in X1HAS1. e, Mutagenesis of channel-lining residues. In vitro HA biosynthesis by the indicated mutants, similar to panel (c). NEC: No Enzyme Control. All experiments (panels c-e) were performed at least in triplicate (n=3 or 8) and error bars represent standard deviations from the means. f, Similar to panel (b) but for channel-lining residues. g, Comparison of HA products obtained from NC64A Chlorella algae infected with PBCV-1 virus, and in vitro synthesized by purified X1HAS1 and CvHAS in LMNG/CHS micelle (det.) and proteoliposome (lip.). The boxed region indicates the hyaluronidase-sensitive isolated sample. H*—hyaluronidase-treated samples.

FIGS. 5A-5F. Substrate binding and primer extension by CvHAS. a, Cross-sectional view of the cryoEM density map for primed CvHAS bound to UDP-GlcA. The priming GlcNAc is colored orange and the UDP-GlcA substrate yellow and cyan for the carbon atoms of the donor sugar and uracil moiety, respectively, as are the densities. b-d, Extension of the GlcNAc primer by GlcA. Densities for GlcNAc and UDP-GlcA, σ=4.5 r.m.s.d (b), HA disaccharide (HA2) and UDP, σ=4.0 r.m.s.d (c) and HA2 only, σ=4.5 r.m.s.d. (d) shown as transparent gray surfaces. e and f, CvHAS active site interactions with GlcNAc and UDP-GlcA. GlcNAc and UDP-GlcA are shown as ball and stick models, while sidechains are shown as sticks only. Inset shows the GlcNAc primer position in the absence of a UDP-GlcA substrate (PDB: 7sp9) as gray sticks.

FIG. 6. Model of HA synthesis and translocation. (1) Binding of a GlcNAc monosaccharide primes HAS. (2) Coordination of UDP-GlcA at the active site repositions the primer to accommodate the donor glycosyl unit, and (3) glycosyl transfer and UDP release generate a disaccharide-bound state with GlcA extending into the catalytic pocket. (4) UDP-GlcNAc binding correlates with disaccharide translocation to position GlcA at the acceptor site. (5 and 6) Glycosyl transfer and UDP release produces a trisaccharide that translocates into the channel after rotation of the non-reducing end terminal GlcNAc unit. The nascent chain likely induces channel opening. (7 and 8) UDP-GlcA binding, glycosyl transfer, and UDP release, followed by rotation of the newly added glycosyl unit generates a tetrasaccharide-bound state. (9 and 10) UDP-GlcNAc binding correlates with translocation of the tetrasaccharide and glycosyl transfer. The GlcNAc terminated chain may translocate into the channel spontaneously, indicated by magenta arrows. Tpr and gray bar: Acceptor site. See Supplementary Discussion for more information.

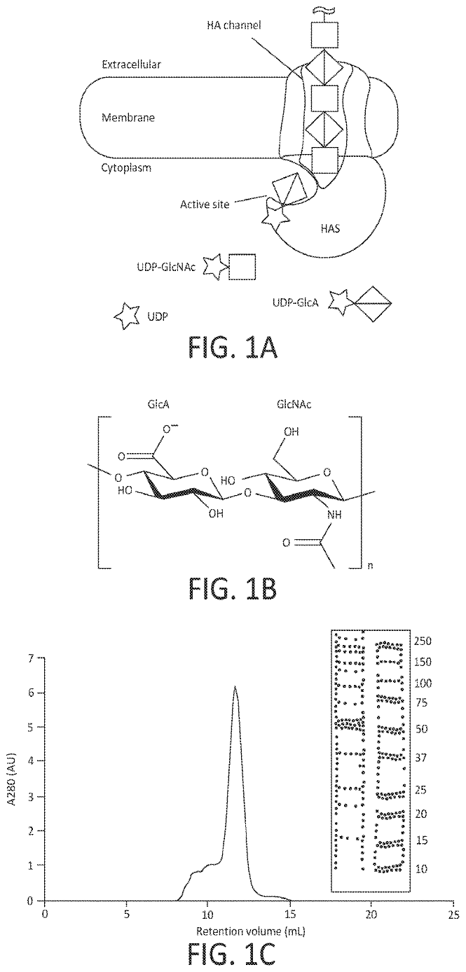

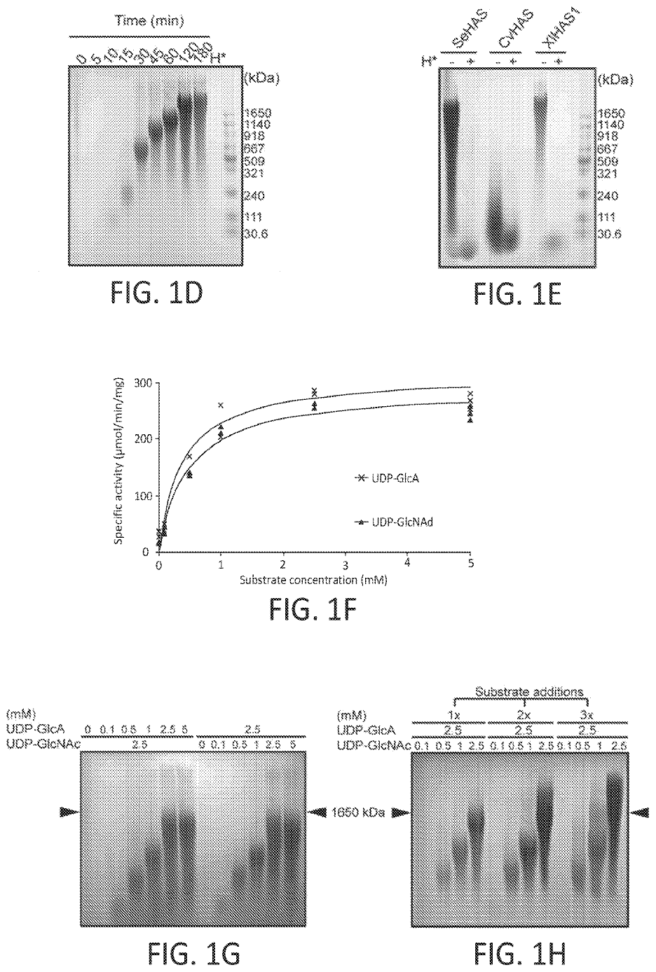

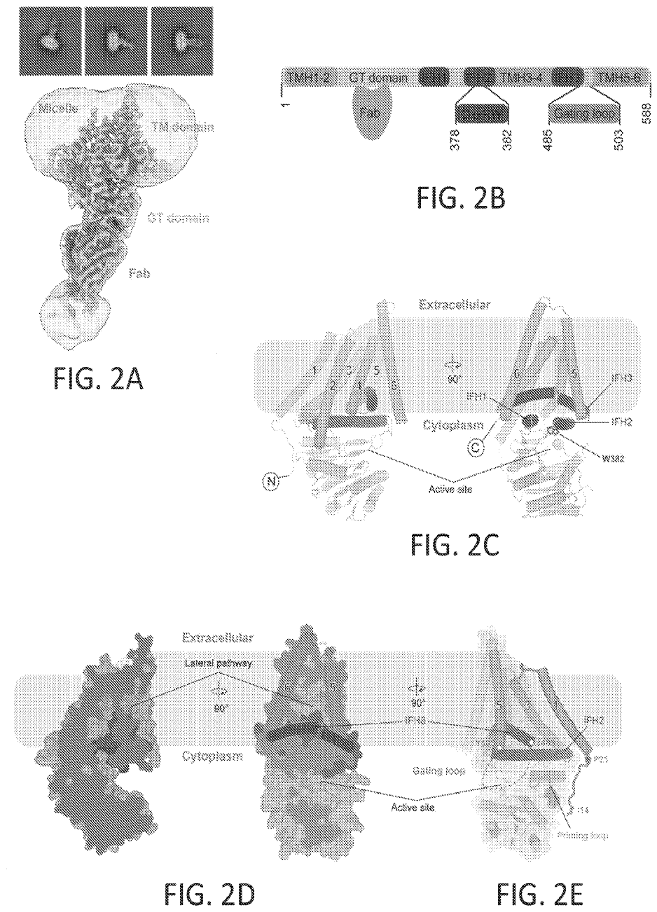

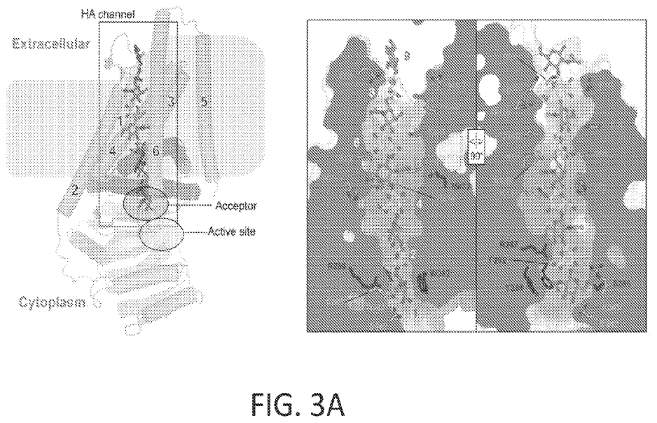

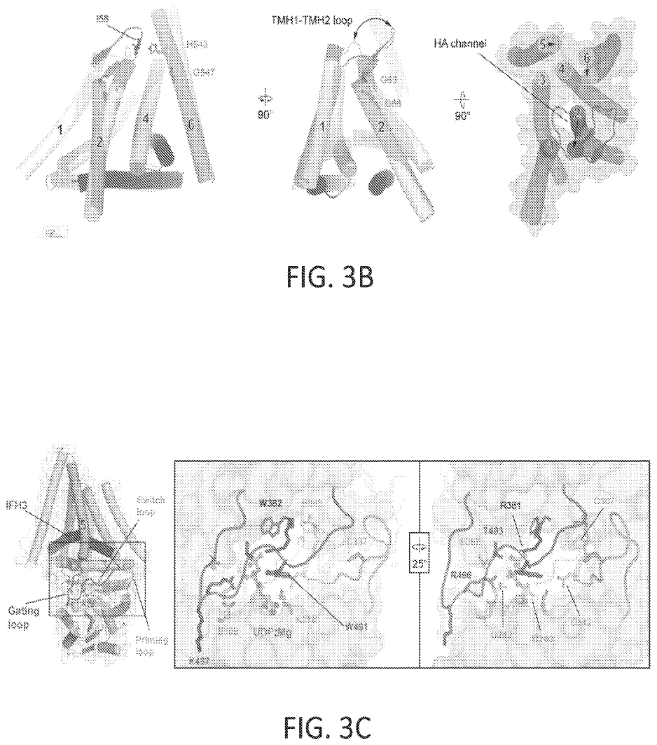

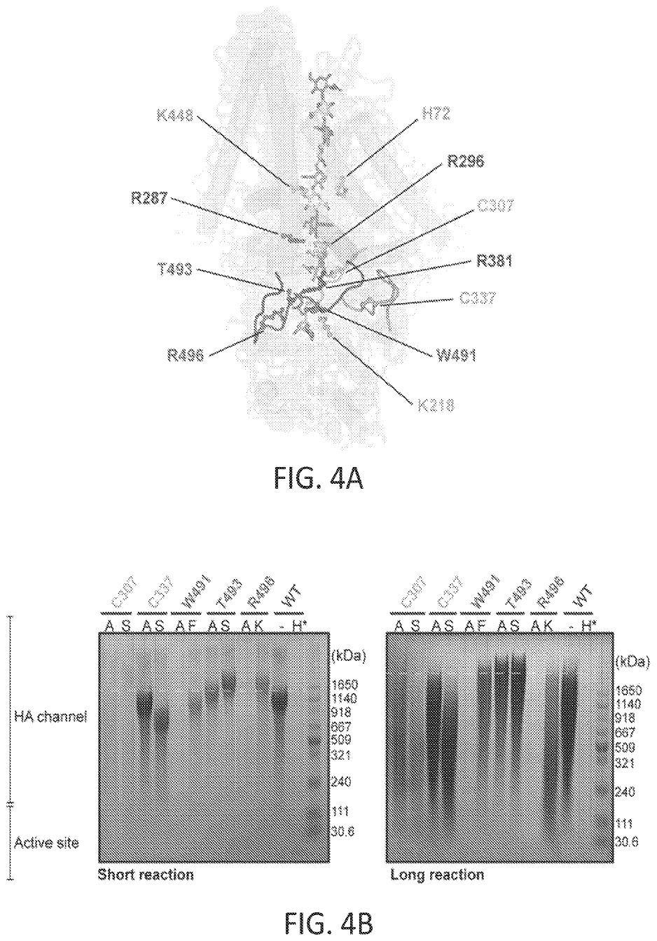

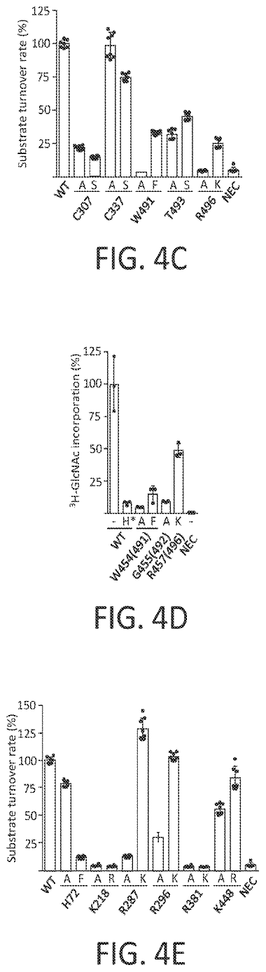

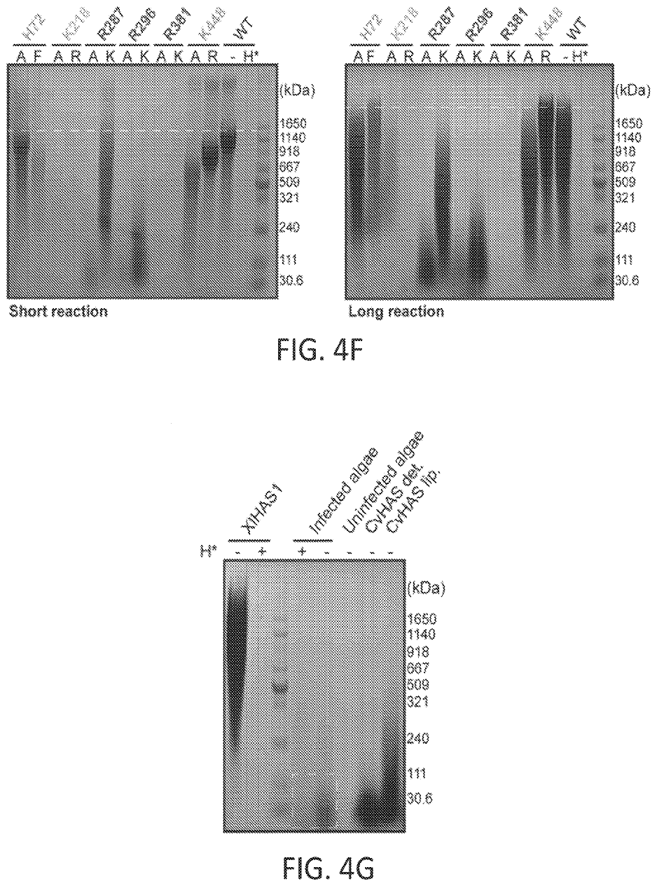

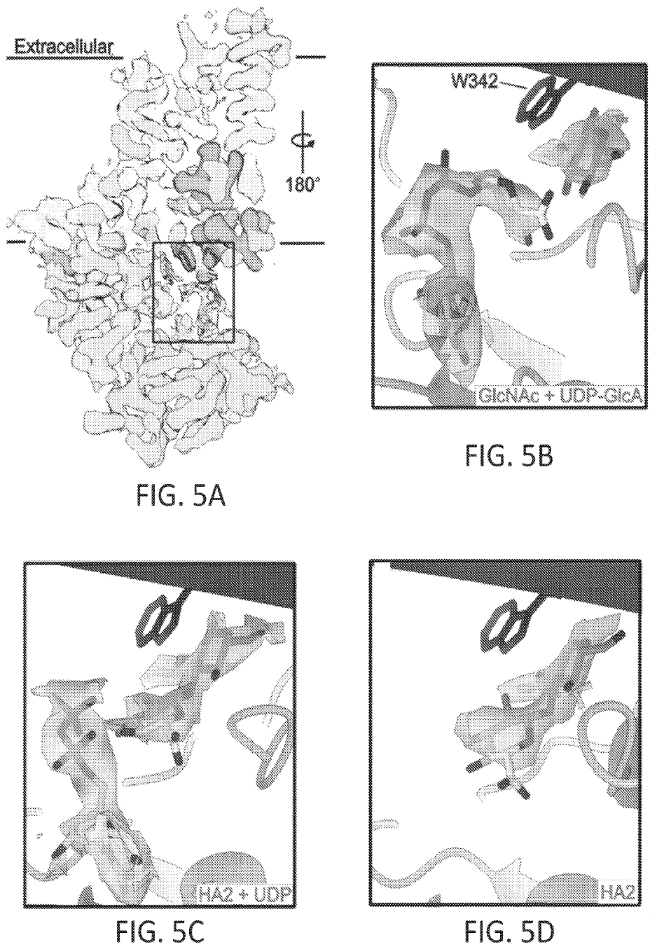

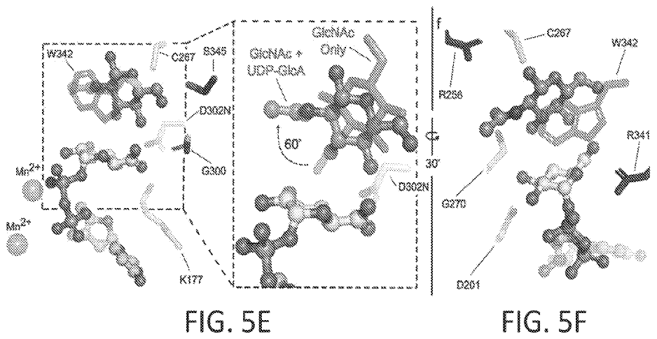

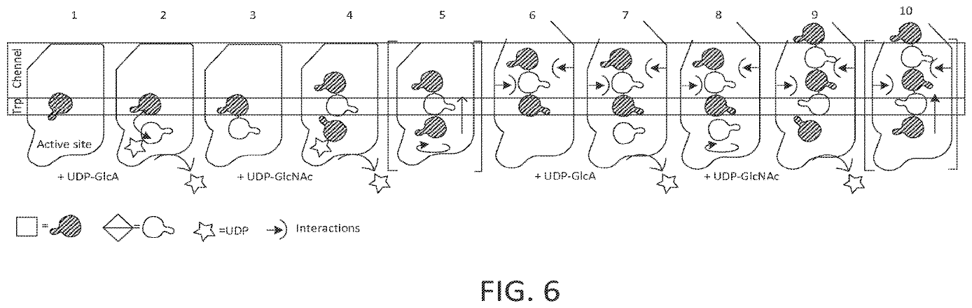

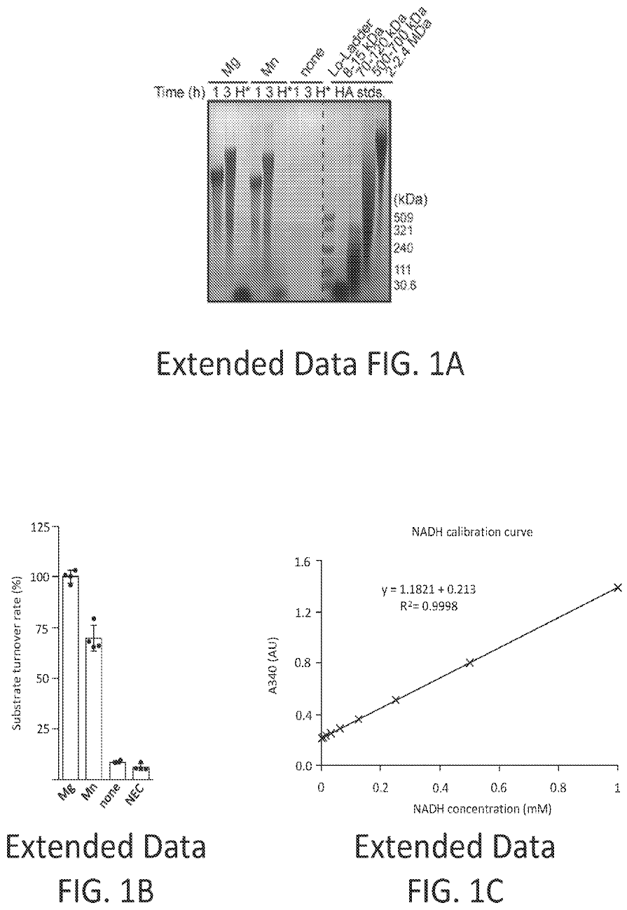

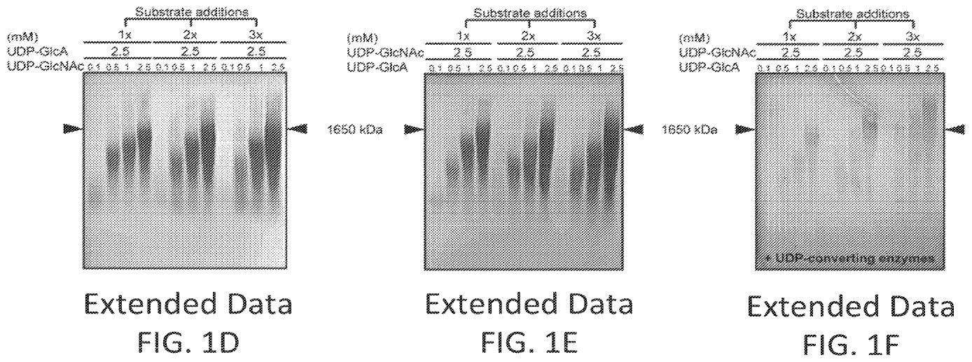

Extended Data FIGS. 1A-1F. In vitro catalytic activity of X1HAS1. a, Agarose gel assay showing HA synthesis is dependent on Mg²⁺, while Mn²⁺ reduces HA production slightly. H*—hyaluronidase digestion. b, Same as panel A but by quantifying UDP release. NEC—no enzyme control. This experiment was performed in quadruplicates (n=4) and error bars represent standard deviations from the means. c, NADH calibration curve used for substrate turnover rate calculations. d, Substrate replenishing in the absence of UDP-converting enzymes (Lactate dehydrogenase—LDH and pyruvate kinase—PK). UDP-GlcA=2.5 mM and UDP-GlcNAc is varied for this assay. e, Same as panel (d), except that UDP-GlcA is varied and UPD-GlcNAc is constant. f, Substrate replenishing with LDH/PK present (UDP-GlcNAc=2.5 mM), reverse to what is shown in FIG. 1h. Arrowheads indicate maximum HA extension without UDP removal and at 2.5 mM of both substrates, corresponding roughly to 1.6 MDa HA marker.

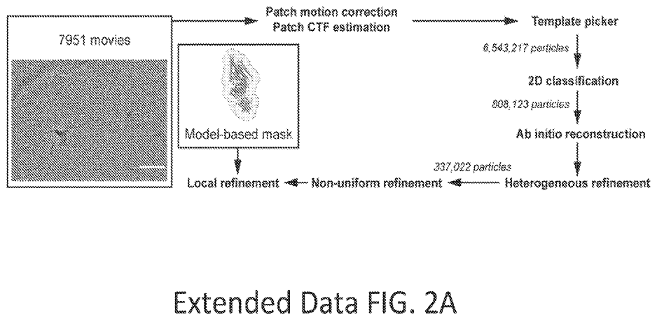

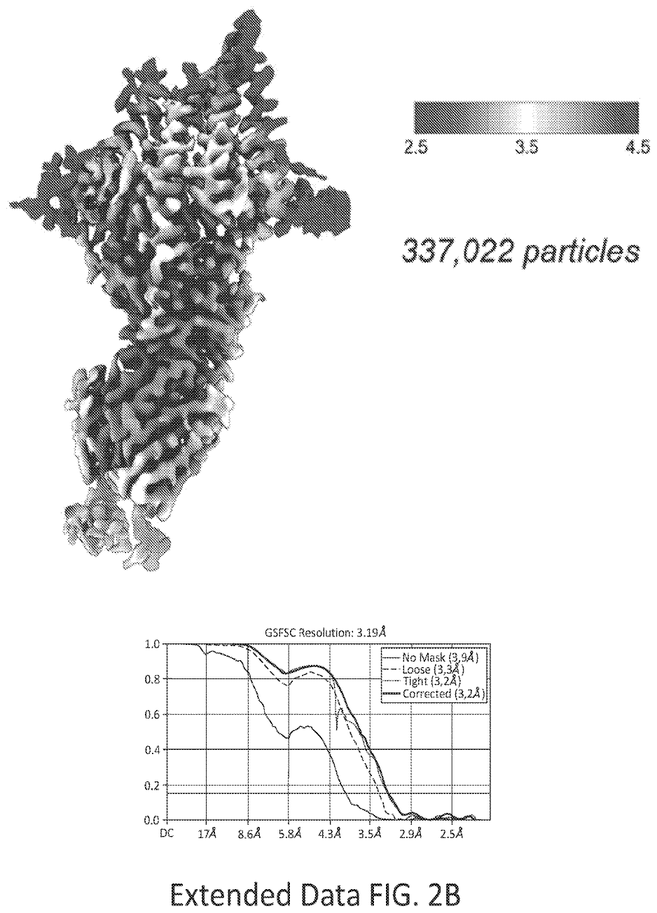

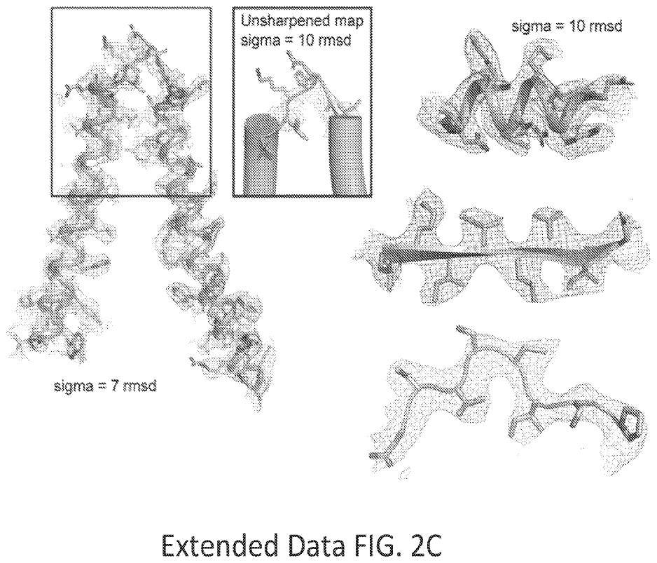

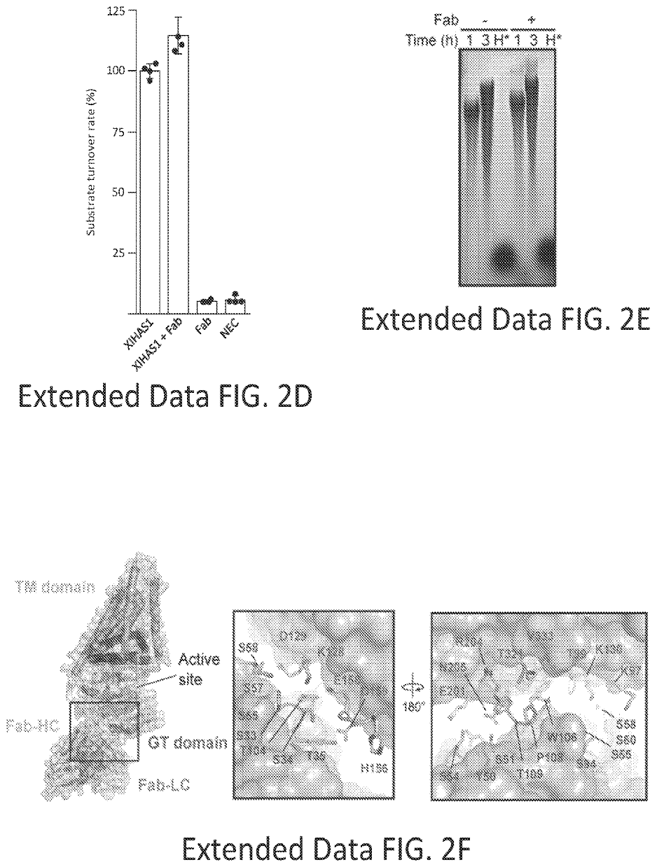

Extended Data FIGS. 2A-2F. (A) CryoEM data processing for apo X1HAS1. a, CryoSPARC workflow for apo XlHAS1. Scale bar on the micrograph corresponds to 100 nm. b, Local resolution of the final map. c, Representative regions showing map quality at the indicated contour levels. d, Substrate turnover rates in the presence and absence of the Fab. This experiment was performed in quadruplicates (n=4) and error bars represent standard deviations from the means. e, HA synthesis in the presence and absence of the Fab. f, HAS-Fab interface. Fab binds the GT domain at a position roughly opposite to the active site.

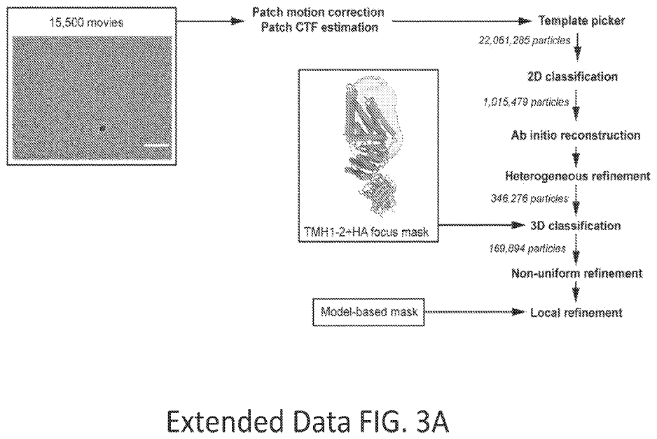

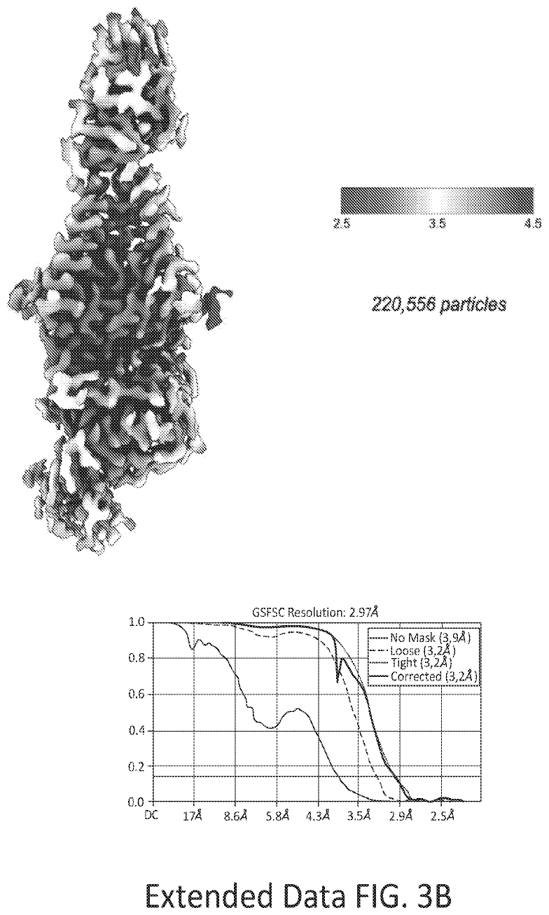

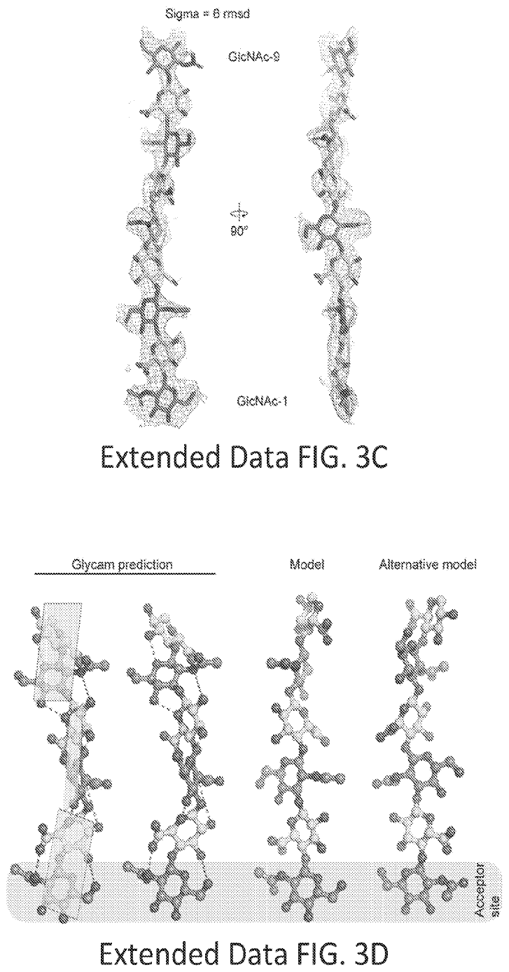

Extended Data FIGS. 3A-3D. CryoEM data processing for HA-bound X1HAS1. a, CryoSPARC workflow for HA-bound X1HAS1. Scale bar on the micrograph: 100 nm. b, Local resolution of the final map. c, Map quality for relevant parts of the model. d, Different HA conformations. Left: Glycam predicted structure of an HA hexasaccharide (glycam.org). Middle: Conformation of HA as modeled in the X1HAS1 complex. Right: Alternative conformation of HA with acetamido and carboxylate groups on opposing sides of the polymer.

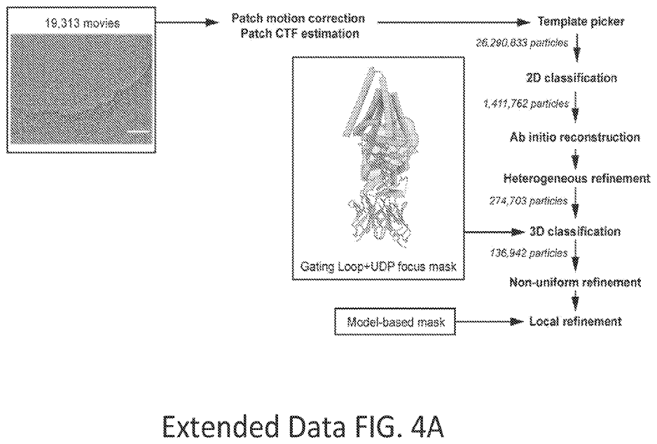

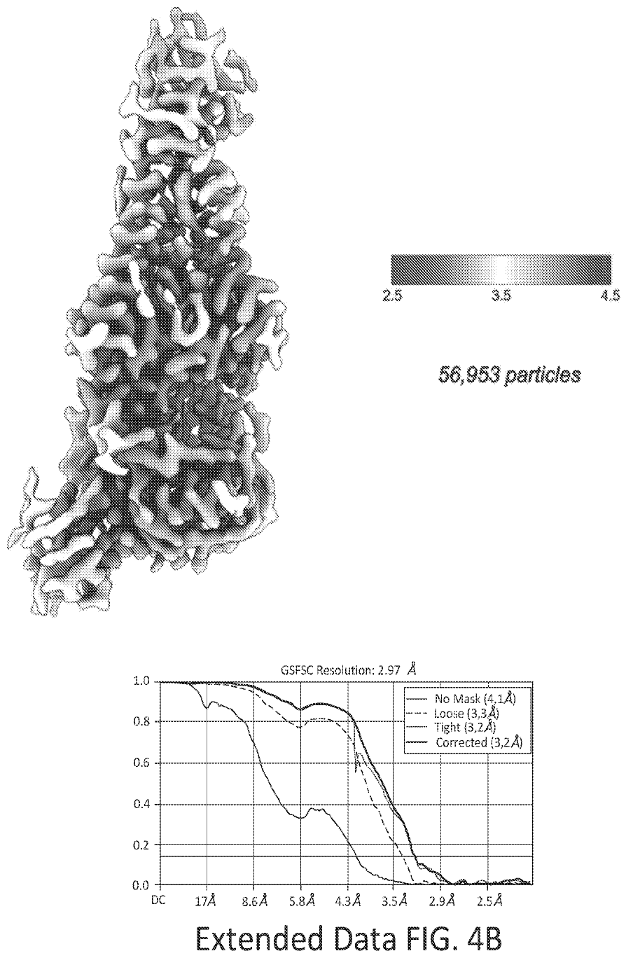

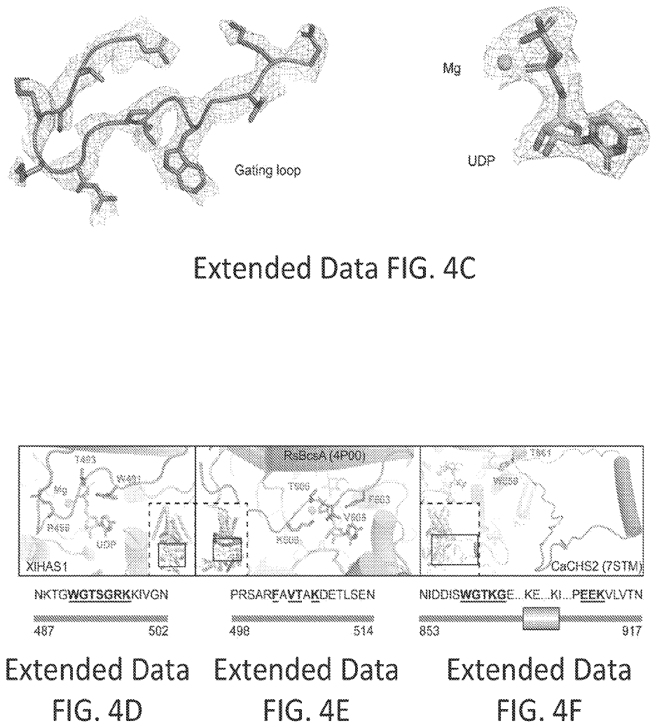

Extended Data FIGS. 4A-4F. CryoEM data processing for UDP-bound X1HAS1. a, CryoSPARC workflow for UDP-bound X1HAS1. Scale bar on the micrograph corresponds to 100 nm. b, Local resolution of the final map. c, Map quality for the gating loop and UDP:Mg²⁺ The map was contoured at σ=7 r.m.s.d. d, Position of the gating loop in UDP-bound X1HAS1. e, Gating loop in RsBcsA (PDB: 4P00). f, Gating loop in Candida albicans CHS2 (PDB: 7STL). The gating loop is shown in deep teal. Numbers indicate gating loop residue ranges. Conserved residues are indicated in bold and underlined font.

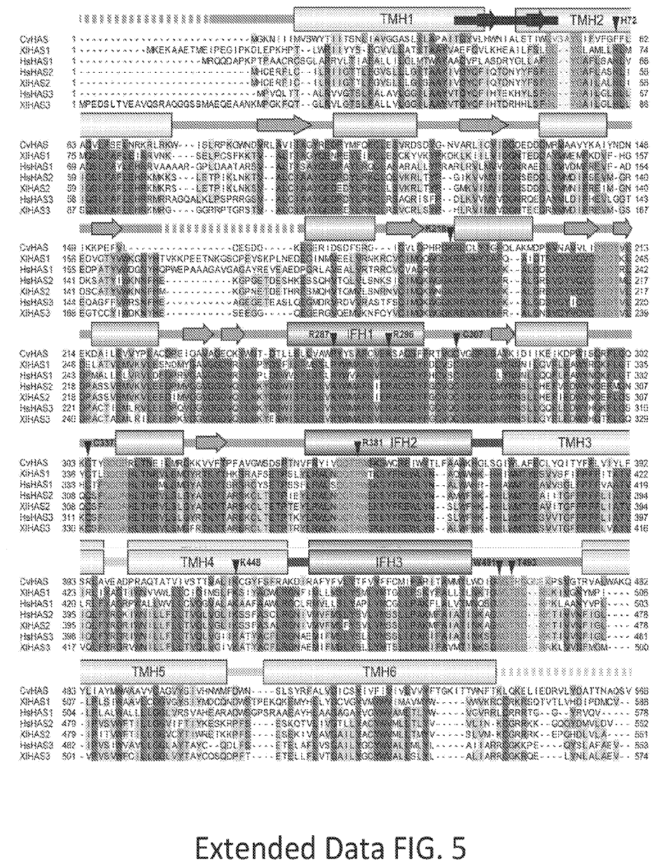

Extended Data FIG. 5. HAS sequence alignments (MUSCLE) (52) showing conserved motifs (GLYG, DxDT, GDDR, QxxRW and WGTSGRK/R, highlighted in yellow), as well as conserved residues used for mutagenesis studies (indicated with arrowheads). The bar on top of the sequences is colored according to structural domains of X1HAS1 (pink: TM domain, purple: TMH1-TMH2 loop, hot pink: IF domain, light blue: GT domain, deep teal: Gating loop). Cylinders indicate α-helices, arrows β-sheets, lines loops, while dashed lines correspond to unstructured regions. (SEQ ID NOS: 1-7)

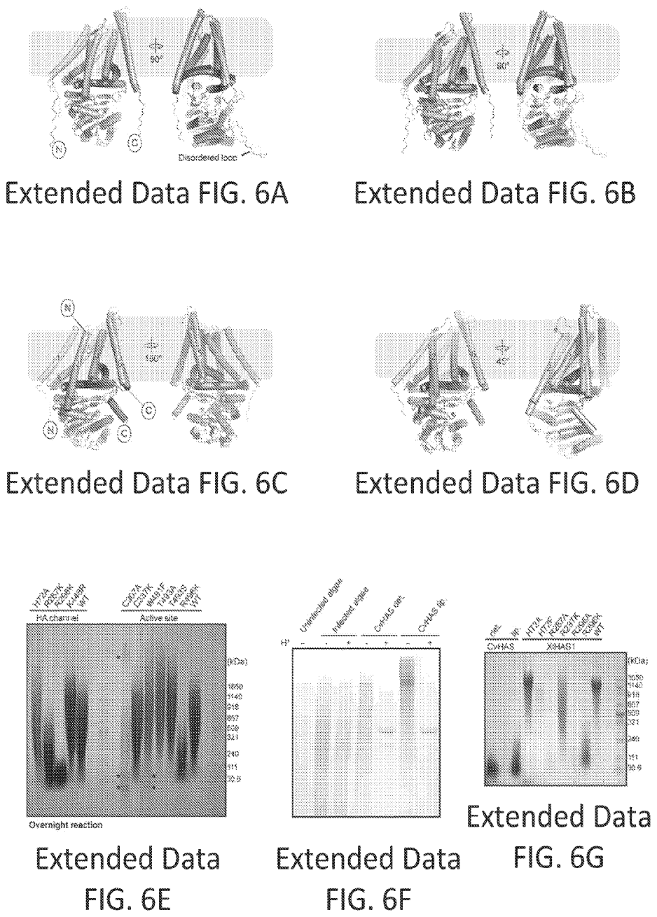

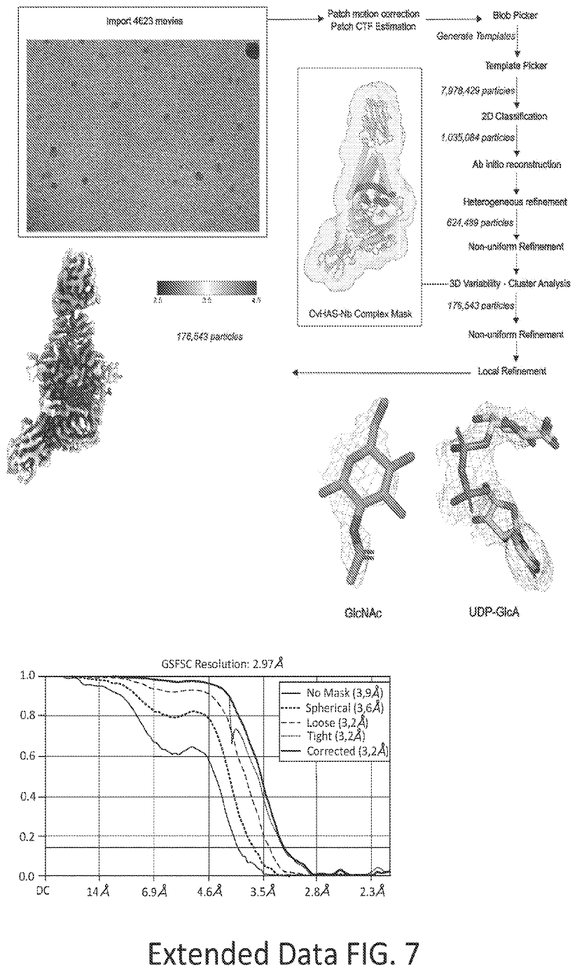

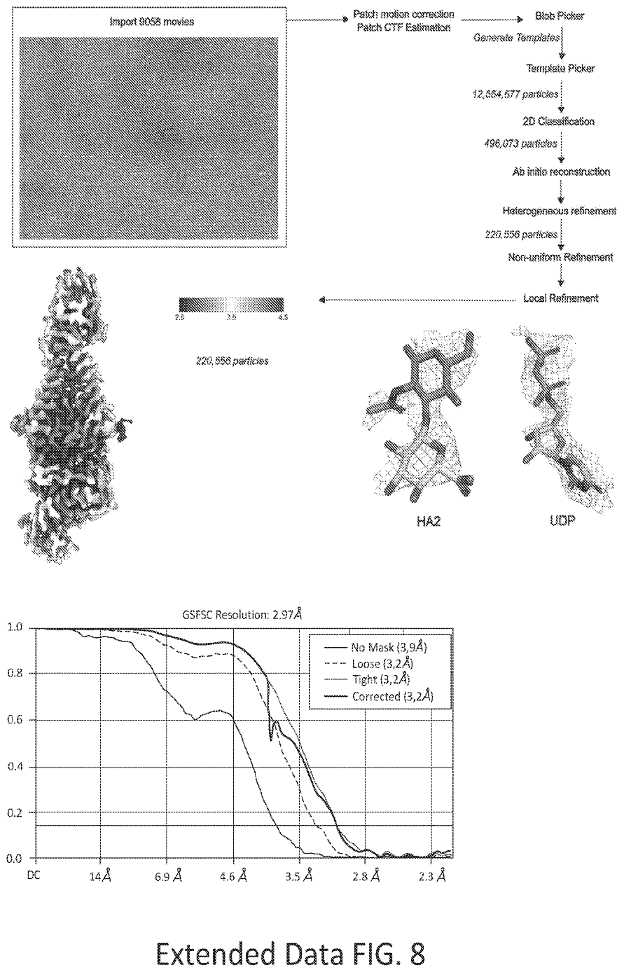

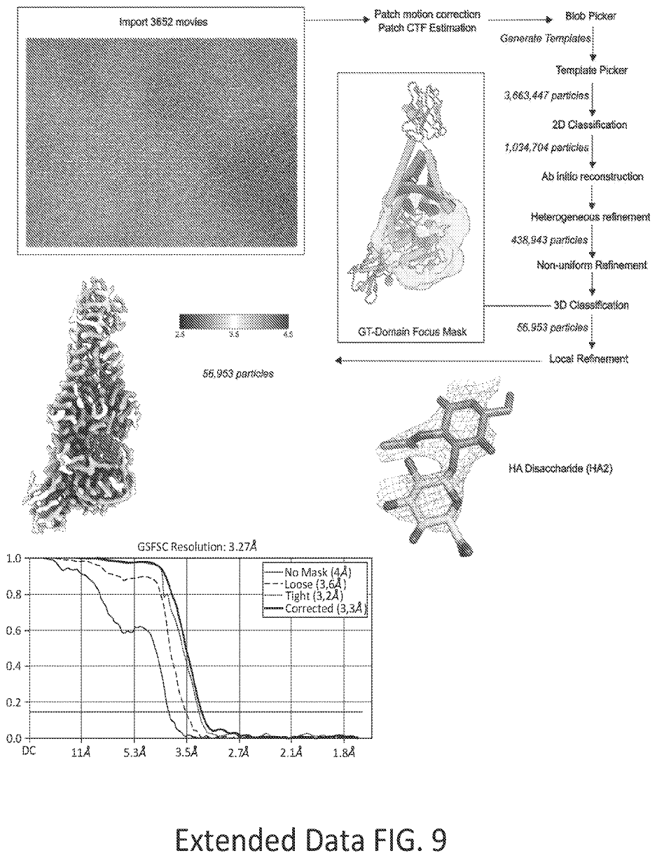

Extended Data FIGS. 6A-6G. HA modeling and gating loop comparison. a, Superimposition of X1HAS1 apo (colored) and a HsHAS1 AlphaFold2 prediction (gray). N- and C-termini of the polypeptide as well as the HAS1-specific but structurally disordered cytoplasmic loop are indicated. b, Superimposition of X1HAS1 HA-bound (colored) and HsHAS1 AlphaFold2 prediction (gray). The AlphaFold2 HsHAS1 model is more similar to the HA-bound structure of X1HAS1. c, Superimposition of X1HAS1 apo (colored) and CvHAS apo (gray, PDB: 7SP6). d, Superimposition of X1HAS1 apo (colored) and CvHAS primed, channel-open state (gray, PDB: 7SPA). e, HA products for selected X1HAS1 mutants visualized on lower percentage agarose gel (0.5 instead of 1%). Reactions were carried out overnight. Asterisks indicate protein bands. f, Comparison of HA products obtained from NC64A Chlorella algae infected with PBCV-1 virus, and in vitro synthesized by purified CvHAS in LMNG/CHS micelle (det.) and proteoliposome (lip.). Samples were analyzed on a 4-20% SDS-PAGE gel scanned in full color to show specific hyaluronidase (H*) degradation of the obtained HA (blue smears) in crude algae extracts. DNA and protein contaminations are present (violet and pink smears/bands). g, Comparison of products synthesized by CvHAS and LMW HA-producing X1HAS1 mutants. Gels shown in panels e-g were stained with Stains-all but only panel (f) is shown in color.

Extended Data FIG. 7. CryoEM data processing for UDP-GlcA-bound CvHAS. CryoEM data processing workflow and map quality for GlcNAc-primed, UDP-GlcA-bound CvHAS. UDP-GlcA and GlcNAc densities are contoured at σ=4.5 r.m.s.d.

Extended Data FIG. 8. CryoEM data processing for disaccharide and UDP-bound CvHAS. CryoEM data processing workflow and map quality for disaccharide (HA2) and UDP-associated CvHAS. HA2 and UDP densities contoured at σ=4.5 r.m.s.d.

Extended Data FIG. 9. CryoEM data processing for disaccharide bound CvHAS. CryoEM data processing workflow and map quality for disaccharide (HA2)-associated CvHAS. HA2 density contoured at σ=4.5 r.m.s.d.

### DETAILED DESCRIPTION

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R.J. Lewis, John Wiley & Sons, New York, N.Y., 2001.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In one embodiment, the first region comprises a first portion and the second region comprises a second portion, whereby, when the first and second portions are arranged in an antiparallel fashion, at least about 50%, including at least about 75%, at least about 90%, or at least about 95% of the nucleotide residues of the first portion are capable of base pairing with nucleotide residues in the second portion. In some embodiments, all nucleotide residues of the first portion are capable of base pairing with nucleotide residues in the second portion.

A “compound,” as used herein, refers to any type of substance or agent that is commonly considered a drug, or a candidate for use as a drug, as well as combinations and mixtures of the above.

A “control” cell is a cell having the same cell type as a test cell. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC5′ share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

As used herein, an “instructional material” includes a publication, a recording, a diagram, or any other medium of expression which can be used to communicate the usefulness of the peptide/protein of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” or protein is one which is produced upon expression of a recombinant polynucleotide.

A “recombinant cell” is a cell that comprises a transgene. Such a cell may be a eukaryotic or a prokaryotic cell. Also, the transgenic cell encompasses, but is not limited to, an embryonic stem cell comprising the transgene, a cell obtained from a chimeric mammal derived from a transgenic embryonic stem cell where the cell comprises the transgene, a cell obtained from a transgenic mammal, or fetal or placental tissue thereof, and a prokaryotic cell comprising the transgene.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

As used herein, a “substantially homologous amino acid sequences” includes those amino acid sequences which have at least about 95% homology, at least about 96% homology, at least about 97% homology, at least about 98% homology, or at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In an example, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO_{4,} 1 mM EDTA at 50° C. with washing in 2× standard saline citrate (SSC), 0.1% SDS at 50° C.; preferably in 7% (SDS), 0.5 M NaPO₄, 1 mM EDTA at 50° C. with washing in 1×SSC, 0.1% SDS at 50° C.; preferably 7% SDS, 0.5 M NaPO₄, 1 mM EDTA at 50° C. with washing in 0.5×SSC, 0.1% SDS at 50° C.; and more preferably in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the present invention.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22: 1859-1862, 1981, and Matteucci et al., J. Am. Chem. Soc. 103:3185, 1981.

As used herein, amino acids are represented by the full name thereof, by the three-letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The term “amino acid” is used interchangeably with “amino acid residue,” and may refer to a free amino acid and to an amino acid residue of a peptide/protein. It will be apparent from the context in which the term is used whether it refers to a free amino acid or a residue of a peptide/protein.

The expression “amino acid” as used herein is meant to include both natural and synthetic amino acids, and both D and L amino acids. “Standard amino acid” means any of the twenty standard L-amino acids commonly found in naturally occurring peptides/proteins. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides/proteins of the present invention, and particularly at the carboxy- or amino-terminus, can be modified by methylation, amidation, acetylation or substitution with other chemical groups which can change the peptide's/protein's circulating half-life without adversely affecting their activity (e.g., peptidomimetic for making peptides protease resistant).

Amino acids have the following general structure:

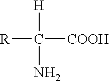

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide/protein compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

As used herein, the term “conservative amino acid substitution” is defined herein as an amino acid exchange within one of the following five groups:
I. Small aliphatic, nonpolar or slightly polar residues:
   Ala, Ser, Thr, Pro, Gly;
II. Polar, negatively charged residues and their amides:
   Asp, Asn, Glu, Gln;
III. Polar, positively charged residues:
   His, Arg, Lys;
IV. Large, aliphatic, nonpolar residues:
   Met Leu, Ile, Val, Cys
V. Large, aromatic residues:
   Phe, Tyr, Trp.

### Hyaluronic Acid

Hyaluronic acid (abbreviated HA; conjugate base hyaluronate), also called hyaluronan, is an anionic, nonsulfated glycosaminoglycan distributed widely throughout connective, epithelial, and neural tissues. It is unique among glycosaminoglycans as it is non-sulfated, forms in the plasma membrane instead of the Golgi apparatus and can be very large.

The average 70 kg (150 lb) person has roughly 15 grams of hyaluronan in the body, one third of which is turned over (i.e., degraded and synthesized) per day.

As one of the chief components of the extracellular matrix, it contributes significantly to cell proliferation and migration, and is involved in the progression of many malignant tumors. Hyaluronic acid is also a component of the group A streptococcal extracellular capsule and may play a role in virulence.

Hyaluronic acid is a major component of the synovial fluid and was found to increase the viscosity of the fluid. Along with lubricin, it is one of the fluid's main lubricating components.

Hyaluronic acid is a component of articular cartilage, where it is present as a coat around each cell (chondrocyte). When aggrecan monomers bind to hyaluronan in the presence of HAPLN1 (hyaluronic acid and proteoglycan link protein 1), large, highly negatively charged aggregates form. These aggregates imbibe water and are responsible for the resilience of cartilage (its resistance to compression). The molecular weight (size) of hyaluronan in cartilage decreases with age, but the amount increases.

A lubricating role of hyaluronan in muscular connective tissues to enhance the sliding between adjacent tissue layers has been suggested. A particular type of fibroblasts, embedded in dense fascial tissues, has been proposed as being cells specialized for the biosynthesis of the hyaluronan-rich matrix. Their related activity could be involved in regulating the sliding ability between adjacent muscular connective tissues.

Hyaluronic acid is also a major component of skin, where it is involved in repairing tissue. When skin is exposed to excessive UVB rays, it becomes inflamed (sunburn), and the cells in the dermis stop producing as much hyaluronan and increase the rate of its degradation. Hyaluronan degradation products then accumulate in the skin after UV exposure.

While it is abundant in extracellular matrices, hyaluronan also contributes to tissue hydrodynamics, movement, and proliferation of cells and participates in a number of cell surface receptor interactions, notably those including its primary receptors, CD44 and RHAMM. Upregulation of CD44 itself is widely accepted as a marker of cell activation in lymphocytes. Hyaluronan's contribution to tumor growth may be due to its interaction with CD44. Receptor CD44 participates in cell adhesion interactions required by tumor cells.

Hyaluronic also plays a role in wound repair, granulation, cell migration and skin healing.

Hyaluronic acid has been FDA-approved to treat osteoarthritis of the knee via intra-articular injection. A 2012 review showed that the quality of studies supporting this use was mostly poor, with a general absence of significant benefits, and that intra-articular injection of HA could possibly cause adverse effects. A 2020 meta-analysis found that intra-articular injection of high molecular weight HA improved both pain and function in people with knee osteoarthritis.

Hyaluronic acid has been used to treat dry eye. Hyaluronic acid is a common ingredient in skin care products. Hyaluronic acid is used as a dermal filler in cosmetic surgery. It is typically injected using either a classic sharp hypodermic needle or a micro-cannula. Some studies have suggested that the use of micro-cannulas can significantly reduce vessel embolisms during injections. Currently, hyaluronic acid is used as a soft tissue filler due to its biocompatibility and possible reversibility using hyaluronidase. Hyaluronan finds use in ophthalmic conditions, for example, first hyaluronan biomedical product, Healon, was developed in the 1970s and 1980s by Pharmacia, and approved for use in eye surgery (i.e., corneal transplantation, cataract surgery, glaucoma surgery, and surgery to repair retinal detachment). Other biomedical companies also produce brands of hyaluronan for ophthalmic surgery. Hyaluronic acid is used to displace tissues away from tissues which are going to be subjected to radiation, for instance in one treatment option for some prostate cancers.

Hyaluronan is used in treatment of articular disorders in horses, in particular those in competition or heavy work. It is indicated for carpal and fetlock joint dysfunctions. It is especially used for synovitis associated with equine osteoarthritis. It can be injected directly into an affected joint, or intravenously for less localized disorders.

Hyaluronic acid is found in cosmetics and skin care formulations, such as creams, lotions, ointments and serums, to, for example, increase hydration, lessen wrinkles, and add fullness.

Incorporation of hyaluronic acid into condiments can enhance the salt perception.

Plants treated with the hyaluronic acid solution exhibits a control rate of 80% or more against plant diseases and a plant growth rate of 20% or more as compared to a control group.

Generally, hyaluronic acid is produced on a large scale by extraction from animal tissues, such as chicken comb, and from Streptococci or other microorganisms.

Hyaluronic acid is synthesized in vivo by a class of integral membrane proteins called hyaluronan synthases, of which vertebrates have three types: HAS1, HAS2, and HAS3. These enzymes lengthen hyaluronan by repeatedly adding D-glucuronic acid and N-acetyl-D-glucosamine to the nascent polysaccharide as it is extruded via ABC-transporter through the cell membrane into the extracellular space. The term fasciacyte was coined to describe fibroblast-like cells that synthesize HA.

Native hyaluronic acid can have a relatively short half-life (shown in rabbits) so various manufacturing techniques have been deployed to extend the length of the chain and stabilize the molecule for its use in medical applications. The introduction of protein-based cross-links, the introduction of free-radical scavenging molecules such as sorbitol, and minimal stabilization of the HA chains through chemical agents such as NASHA (non-animal stabilized hyaluronic acid) are all techniques that can be used to preserve its shelf life.

Hyaluronic acid synthesis has been shown to be inhibited by 4-methylumbelliferone (hymecromone), a 7-hydroxy-4-methylcoumarin derivative.

### Example I

### Introduction

Hyaluronan (HA) is an extracellular matrix polysaccharide of vertebrate tissues, with functions in osmoregulation, cell signaling and joint lubrication (1, 2). HA's physiological functions are influenced by its length and gel-forming properties (3). Healthy tissues usually contain polymers of several megadaltons in size (2). Inflammatory processes and osteoarthritis, however, associate with a decline in HA length to hundreds of kilodaltons (4, 5). Humans express three HAS isoforms, with HAS2 being essential (6). While HAS1 and HAS2 produce HA polymers of several megadaltons, HAS3 derived products usually range from 105 to 106 Da in size (7).

HA consists of alternating N-acetylglucosamine (GlcNAc) and glucuronic acid (GlcA) units (8) and is synthesized and secreted by HA-synthase (HAS), a membrane-embedded processive glycosyltransferase (GT) (FIG. 1A, B) (9-11). HAS integrates several functions: first, binding its substrates (uridine diphosphate (UDP)-activated GlcA and GlcNAc); second, catalyzing glycosyl transfer; and third, translocating the nascent polymer across the plasma membrane. How these functions are integrated for HA biosynthesis is poorly understood.

While HA is ubiquitously expressed in vertebrates, the Paramecium bursaria Chlorella virus (Cv) encodes a HAS similar to the vertebrate homologs (12). Structural and functional analyses of CvHAS provided insights into the enzyme's architecture and how it initiates HA biosynthesis (13). However, because the enzyme produces HA polymers substantially shorter than vertebrate HAS, as well as lacking a nascent HA chain in structural analyses, little was learned about how HAS coordinates the nascent HA polymer, translocates it between elongation steps, and regulates its size.

Biochemical and structural analyses of Xenopus laevis HAS isoform 1 (X1HAS1, formerly DG42 (14-16)) address these aspects of HA biosynthesis. In vitro, X1HAS1 produces polysaccharides similar in size to natively expressed HA. Cryogenic electron microscopy (cryoEM) analyses of the enzyme bound to a nascent HA chain reveal how it creates an HA translocation channel and coordinates the polysaccharide during secretion. Complementary cryoEM structures of CvHAS bound to the substrate UDP-GlcA and reaction products delineate the mechanism of alternating substrate polymerization and suggest a model for HA translocation. Further, site-directed mutagenesis of HA-coordinating residues/channel lining residues identifies interactions modulating HA length.

### Materials and Methods

### X1HAS1 Expression

The synthetic gene encoding N-terminally dodeca-histidine-tagged X1HAS1 (Uniprot ID: P13563) was cloned into the pACEBac-1 vector using BamHI and HindIII restriction sites. X1HAS1 mutants were generated using QuickChange and Q5 SDM methods (35, 36). Cloning was confirmed by restriction analyses and DNA sequencing. Baculoviruses harboring each target gene were prepared as described (31). Briefly, the X1HAS1 containing pACEBac-1 plasmid was transformed into E. coli DH10MultiBac cells. Bacmids for the wild type and all X1HAS1 mutants were purified from 3 white colonies and transfected into Sf9 cells at 1×10⁶ cells/mL using the FuGene reagent (Promega). Cells were maintained in ESF921 medium (Expression Systems) at 27° C. with mild shaking. The baculovirus was amplified to generate P2 virus stock, which was used at 1.5% culture volume to infect Sf9 cells at a density of 3×10⁶ cells/mL. Cells grown in 1 L media bottles (0.45 L per bottle) were pelleted by centrifugation 48 h post-infection and resuspended in 40 mL of Buffer A (40 mM TrisHCl pH 7.8, 150 mM NaCl, 10% glycerol, 5 mM MgCl₂) per 0.9 L of cell culture. The harvested cells were flash-frozen and stored at −80° C. for subsequent use.

Example of a hyaluronan synthase gene/protein from Xenopus laevis is:

>NM_001086227.1-gene
(SEQ ID NO: 8)
gaattcaaaaataagctcctgctttgaggccactggggagcaacatccaaggggttggggagcaacatgtta
ctcacgagctactggttggggatcactgacctaaggTATTCACCCCAAGGTCCGACCAAAGACTCATTGGGC
CTAGCTTTGCCAACAAGGGGTAATTGTGGTTGTTCCATAGTGGGATTATGAAACATTTCAACCCAAAATCTT
CTCCTGTGttaaatttgataaaaatatcggtaaaaaaaaaagcccccagagtatcacaatttttatttccta
ttacatttatttgttacttttcaaattaaaaataagagGTTCCAGGTCAGGGCTGCACCAATCACCAAGCAA
TGCTCCCTCCCCTCATTTGCTATGGAGTGGCATTCCAGCCTATTGTTATGCAGGGG
TGTGGCCTATACAGGAGCCTGCAGGTATATAAACCCAGGTCAGAGTGGTCATTGT
CACTAAAAACTTGGCATATCCCATGTGTGTGTCTTGGGGCCTCTTCATTCGGCAA
CATGAAGGAAAAAGCTGCAGGTAAGTGTCTTTTTCTGAACCTTTTTATAATTCCTT
TATTCACCCCCCCCCAACGCATAGGATTTTAGGAGacccacattttaaagggaaaatacaccccAA
ATCTTTTTTATGCTTAATGAATGAATTTAAAGTTATATCTAAGTGTAATTGCACTA
GAATGCTGCATTGGTATTGGAAACTTGcctataattaattttattgggGTGGAGTTCCCCATTA
AATTGTGCCTTGTTACTATTCTCTTATTTGACTGTTCTTACACATAATTTCATATTC
ACTAAAACATAATCCACGTTATTATAAGGCAAATTTATAAATGCCAAATTGTTTCTCTTAGtcagtacagta
taagggtatagcttattgtgtgcccagaacattccttctctgtatatttgtatttatacatatgggaggagga
ggtgccatattgattcccttagacagtacagtataagggtatagcttattgtgtgcccagaacattccttctc
tgtatatttgtatttatacatatgggaggaggtgccatattgattcccttagacagtacagtatgagggtata
gcttattgtgtgcccagaacattccttctctgtatatttgtatttatacatatgggaggaggtgccatattga
ttcccttacacagtacagtatgagggtatagcttattgtgtgcccagaacattccttctctgtatatttgtat
ttatacatatgggaggaggaggtgccatattgattccctagacagtacagtatgagggtatagtttattgtgt
gcccggaacattctttctctgtatatttgtatttatacatatgggaggaggaggtgccctattgattccctta
gaccatACAGTATGAGGgaacagcttattgtgtgcccagagcattcctacTCTGTATATGTAACAGTTTATCC
ATAATTTCTAatccttctctttttctttccttagAAACAATGGAGATTCCTGAAGGGATCCCCAAAGATCTAG
AGCCAAAACACCCCACCCTTTGGAGGATAATTTATTATTCTTTTGGTGTGGTGCTATTAGCTACCA
TTACAGCAGCCTATGTGGCAGAGTTCCAGGTCCTCAAACATGAAGCCATTCTCTT
CTCCCTTGGGCTTTATGGTCTTGCAATGCTTCTCCACCTGATGATGCAGAGCCTCT
TTGCCTTCCTGGAGATACGCAGGGTAAATAAGAGTGAGCTTCCTTGCAGCTTTAA
GAAGACAGTGGCTCTGACCATTGCTGGGTATCAGGAGAACCCTGAGTACCTGAT
AAAGTGCTTGGAATCCTGCAAGTATGTGAAATACCCCAAAGATAAACTCAAGAT
CATTTTGGTCATCGATGGGAACACAGAGGATGATGCCTACATGATGGAGATGTTC
AAAGACGTGTTCCACGGTGAAGATGTAGGCACCTACGTATGGAAGGGAAATTAC
CACACTGTTAAAAAGCCTGAGGAGACCAATAAGGGATCCTGTCCTGAGGTTTCT
AAGCCCTTGAATGAAGATGAAGGTATCAATATGGTGGAAGAGCTTGTTAGAAAC
AAGAGATGTGTGTGCATCATGCAACAGTGGGGCGGAAAAAGAGAGGTCATGTAC
ACAGCATTCCAGGCCATTGGGACTTCTGTGGACTATGTACAGGTGGGTCTATTTA
TCTTTCAAAAATGGCCATGGTAGAGTCTGATTCCCTTATTTATGTTCTCAGCATGT
TCTCCTCCGATGTTGTGCTTCTTCCTTTTATTTGCCTCTTTGGATCTTCTTGTCCAT
TCATGTTATGTTGTTATTCATGTGTTTCATCTTTTAATTCCATTAATTATAGGTCTG
TGACTCGGACACCAAACTGGATGAACTGGCAACAGTGGAAATGGTGAAGGTTCT
GGAGTCCAATGACATGTACGGCGCAGTGGGAGGAGACGTTCGCATTCTGAACCC
TTATGATTCCTTCATTAGTTTCATGAGCAGCCTGCGTTACTGGATGGCGTTTAACG
TGGAGAGGGCCTGCCAGTCTTACTTCGACTGCGTGTCCTGTATAAGTGGACCTCT
GGGTAGGTGGcccaaaaatattatataacttTGGTTCCATGTAATGGATCTTGTTGCCATGTGT
ATCATAATCGCAAGCTCATCATTACATTTATGTTCCCCTATTTGAAGGAATGTAC
CGGAACAACATTCTCCAGGTGTTTTTGGAAGCCTGGTACAGACAGAAATTTTTGG
GAACCTATTGTACTTTGGGAGATGATAGACATCTGACAAACCGAGTGCTCAGCAT
GGGATATCGCACCAAGTAAGTTGTCTGTGAATGTGGTGAGCAATTCTATGCGGGG
AAAGCAGTGGGACAGATTGTGACTTTTCATCTGATGATCTCTCTACGTCTTTCTCT
ATTACTCAACATGACCAATAATTGACTTATTTGTCCCTTTCTCTCTTTAAGATACA
CCCACAAATCCAGAGCATTCTCCGAAACTCCATCCCTGTATCTCCGGTGGTTGAA
CCAGCAAACCCGGTGGACCAAGTCCTACTTCCGAGAGTGGCTGTATAATGCCCagt
ggtggcacaagcatcacaTCTGGATGACCTATGAGTCTGTGGTGTCCTTCATCTTTCCCTTC
TTCATCACTGCCACTGTTATCCGCCTCATCTATGCCGGCACCATATGGAATGTTGT
GTGGCTCCTCCTGTGCATCCAGATCATGTCTCTCTTCAAATCCATCTATGCCTGCT
GGCTCCGCGGCAACTTCATTATGCTCCTGATGTCTCTCTACTCCATGCTGTACATG
ACTGGGCTTCTGCCATCCAAGTACTTTGCCTTGTTGACCTTAAACAAGACCGGTT
GGGGAACATCTGGGCGCAAGAAGATAGTAGGCAACTACATGCCAATACTGCCCC
TGTCCATATGGGCAGCTGTTCTGTGTGGAGGGGTGGGTTATAGTATCTATATGGA
CTGTCAAAATGACTGGAGCACCCCTGAAAAGCAAAAGGAGATGTACCATCTATT
GTATGGGTGTGTGGGCTATGTAATGTATTGGGTAATCATGGCTGTGATGTACTGG
GTCTGGGTGAAGAGGTGCTGCAGGAAGAGGTCCCAAACTGTCACCCTGGTCCAT
GACATTCCTGATATGTGTGTTTAAAGGAATACTGCTATCACACGATCTATAGTCC
CTTTCTATTATTGCAACCTATGTGTATTTTACTGTCAATAGGTGTCATCATCATCT
ATGGCTACCTGCTCCATAACAAAACCATGAAATCTATAGCCAGCAACAAGAAGC
GGCGACTATTATTTTCATTCTCATATTGAGAGACGGTTTTTGTCTCCTGGTTTTTA
TCCAGTCTCCAATATATCACAAAAAAGGTTTTCAAGTTATTAATAAtgaaattgctattaaa
agcagcttcTGTCCTTTGCGATATCTTTCTCCGTTTgacagaaacaatgtagcaataatGAGACCCTT
AatactccagttcccacaatgccttgttcACTTGGTAACCCCCTTCTTCTCAGGTATGTTAATCACC
AAACATGCAAGGTATCATGGGAATTAGGTCCTTGCTTGAGGAGGTTGGACTGCTA
ATACAAtgtagcaattgcatttacatttaaCTTTAATATCACTGACAAAATTCTATGTATATTGGG
GAAAATTGCCTTTAAGACAGTTTATATTGCtcaagtgtttttatttacattttttattcctaCAAATATAT
CATAAATCTCTCCCTAATCCCCTGTTGCTGtgatgttaataaagttatgtttcCTTGTACTGTAA
(SEQ ID NO: 9)
MKEKAAETMEIPEGIPKDLEPKHPTLWRIIYYSFGVVLLATITAAYVAEFQVLKHEAI
LFSLGLYGLAMLLHLMMQSLFAFLEIRRVNKSELPCSFKKTVALTIAGYQENPEYLIK
CLESCKYVKYPKDKLKIILVIDGNTEDDAYMMEMFKDVFHGEDVGTYVWKGNYHT
VKKPEETNKGSCPEVSKPLNEDEGINMVEELVRNKRCVCIMQQWGGKREVMYTAF
QAIGTSVDYVQVCDSDTKLDELATVEMVKVLESNDMYGAVGGDVRILNPYDSFISF
MSSLRYWMAFNVERACQSYFDCVSCISGPLGMYRNNILQVFLEAWYRQKFLGTYCT
LGDDRHLTNRVLSMGYRTKYTHKSRAFSETPSLYLRWLNQQTRWTKSYFREWLYN
AQWWHKHHIWMTYESVVSFIFPFFITATVIRLIYAGTIWNVVWLLLCIQIMSLFKSIY
ACWLRGNFIMLLMSLYSMLYMTGLLPSKYFALLTLNKTGWGTSGRKKIVGNYMPIL
PLSIWAAVLCGGVGYSIYMDCQNDWSTPEKQKEMYHLLYGCVGYVMYWVIMAVM
YWVWVKRCCRKRSQTVTLVHDIPDMCV
mRNA/CDS:
(SEQ ID NO: 10)

| | |
|---|---|
| 1 | gaattcaaaa ataagctcct gctttgaggc cactggggag caacatccaa ggggttgggg |
| 61 | agcaacatgt tactcacgag ctactggttg gggatcactg acctaaggta ttcaccccaa |
| 121 | ggtccgacca aagactcatt gggcctagct ttgccaacaa ggggtaattg tggttgttcc |
| 181 | atagtgggat tatgaaacat ttcaacccaa aatcttctcc tgtgttaaat ttgataaaaa |
| 241 | tatcggtaaa aaaaaagccc ccagagtatc acaattttta tttcctatta catttatttg |
| 301 | ttacttttca aattaaaaat aagaggttcc aggtcagggc tgcaccaatc accaagcaat |
| 361 | gctccctccc ctcatttgct atggagtggc attccagcct attgttatgc aggggtgtgg |
| 421 | cctatacagg agcctgcagg tatataaacc caggtcagag tggtcattgt cactaaaaac |
| 481 | ttggcatatc ccatgtgtgt gtcttggggc ctcttcattc ggcaacatga aggaaaaagc |
| 541 | tgcagaaaca atggagattc ctgaagggat ccccaaagat ctagagccaa aacaccccac |
| 601 | cctttggagg ataatttatt attcttttgg tgtggtgcta ttagctacca ttacagcagc |
| 661 | ctatgtggca gagttccagg tcctcaaaca tgaagccatt ctcttctccc ttgggcttta |
| 721 | tggtcttgca atgcttctcc acctgatgat gcagagcctc tttgccttcc tggagatacg |
| 781 | cagggtaaat aagagtgagc ttccttgcag ctttaagaag acagtggctc tgaccattgc |
| 841 | tgggtatcag gagaaccctg agtacctgat aaagtgcttg gaatcctgca agtatgtgaa |
| 901 | ataccccaaa gataaactca agatcatttt ggtcatcgat gggaacacag aggatgatgc |
| 961 | ctacatgatg gagatgttca aagacgtgtt ccacggtgaa gatgtaggca cctacgtatg |
| 1021 | gaagggaaat taccacactg ttaaaaagcc tgaggagacc aataagggat cctgtcctga |
| 1081 | ggtttctaag cccttgaatg aagatgaagg tatcaatatg gtggaagaac ttgttagaaa |
| 1141 | caagagatgt gtgtgcatca tgcaacagtg gggcggaaaa agagaggtca tgtacacagc |
| 1201 | attccaggcc attgggactt ctgtggacta tgtacaggtc tgtgactcgg acaccaaact |
| 1261 | ggatgaactg gcaacagtgg aaatggtgaa ggttctggaa tccaatgaca tgtacggcgc |
| 1321 | agtgggagga gacgttcgca ttctgaaccc ttatgattcc ttcattagtt tcatgagcag |
| 1381 | cctgcgttac tggatggcgt ttaacgtgga gagggcctgc cagtcttact tcgactgcgt |
| 1441 | gtcctgtata agtggacctc tgggaatgta ccggaacaac attctccagg tgtttttgga |
| 1501 | agcctggtac agacagaaat ttttgggaac ctattgtact ttgggagatg atagacatct |
| 1561 | gacaaaccga gtgctcagca tgggatatcg caccaaatac acccacaaat ccagagcatt |
| 1621 | ctccgaaact ccatccctgt atctccggtg gttgaaccag caaacccggt ggaccaagtc |
| 1681 | ctacttccga gagtggctgt ataatgccca gtggtggcac aagcatcaca tctggatgac |
| 1741 | ctatgagtct gtggtgtcct tcatctttcc cttcttcatc actgccactg ttatccgcct |
| 1801 | catctatgcc ggcaccatat ggaatgttgt gtggctcctc ctgtgcatcc agatcatgtc |
| 1861 | tctcttcaaa tccatctatg cctgctggct ccgcggcaac ttcattatgc tcctgatgtc |
| 1921 | tctctactcc atgctgtaca tgactgggct tctgccatcc aagtactttg ccttgttgac |
| 1981 | cttaaacaag accggttggg gaacatctgg gcgcaagaag atagtaggca actacatgcc |
| 2041 | aatactgccc ctgtccatat gggcagctgt tctgtgtgga ggggtgggtt atagtatcta |
| 2101 | tatggactgt caaaatgact ggagcacccc tgaaaagcaa aaggagatgt accatctatt |
| 2161 | gtatgggtgt gtgggctatg taatgtactg ggtaatcatg gctgtgatgt actgggtctg |
| 2221 | ggtgaagagg tgctgcagga agaggtccca aactgtcacc ctggtccatg acattcctga |
| 2281 | tatgtgtgtt taaaggaata ctgctatcac acgatctata gtcccttcta ttgttgcaac |
| 2341 | ctatgtgtat tttactgttc aataggtgtc atcatcatct atggctacct actccataac |
| 2401 | aaaaccatag aatctatagc cagcaacaag aagcggcgac tattattttc attctcatat |
| 2461 | tgagagacgg tttttgtctc ctggttttta tccagtctcc aatatatcac aaaaaaggtt |
| 2521 | ttcaagttat taataatgaa attgctatta aaagcagctt ctgtctgttc tttgcgatat |
| 2581 | ctttctccgt ttgacagaaa caatgtagca ataatgagac ccttaatact ctagttccca |
| 2641 | caatgccttg ttcacttggt aatccccttc ttctcaggta tgttaatcac caaacatgca |
| 2701 | aggtatcatg ggaattaggt ccttgcttga ggaggttgga ctgctaatac aatgcagcaa |
| 2761 | ttgcatttac atttaacttt aatatcactg acaaaattct atgtatattg gggaaaattg |
| 2821 | cctttaagac agtttatatt gctcaagtgt ttttatttac attttttatt cctacaaata |
| 2881 | tatcataaat ctctccctaa tcccctgttg ctgtgatgtt aataaagtta tgtttccttg |
| 2941 | tactgtaaaa |

### CvHAS Expression

Expression of CvHAS was performed as described (13). Briefly, electrocompetent E. co/i C43 cells were transformed with a pET28α-CvHAS expression vector. LB medium supplemented with 50 μg/mL kanamycin was inoculated with the transformed C43 cells and grown overnight in an orbital shaker at 37° C., 220 rpm. The overnight culture was used to inoculate 4 L of terrific broth supplemented with 1×M salts (25 mM Na₂HPO₄, 25 mM KH₂PO₄, 50 mM NH₄Cl, 5 mM Na₂SO₄) (37) and 50 μg/mL kanamycin. Cultures were grown at 30° C. with 220 rpm shaking to OD600=0.8, then cooled to 20° C. for 1 hour. To induce expression, isopropyl-β-D-thiogalactopyranoside was added to a final concentration of 100 μg/mL. After 18 hours, cells were harvested by centrifugation at 5000 rpm in a JLA-8.1000 rotor for 20 minutes. Cell pellets were flash-frozen in liquid nitrogen before storage at −80° C.

### X1HAS1 Purification

All preparation steps were carried out at 4° C. unless stated otherwise. To purify X1HAS1, typically 0.9 L-culture worth of cell suspension was thawed and diluted to 200 mL using Buffer A supplemented with 5 mM β-ME, 1 mM phenylmethylsulfonyl fluoride (PMSF), 10 mM imidazole, 1% β-dodecyl maltoside (DDM) and 0.2% cholesteryl hemisuccinate (CHS). Cells were lysed using a tissue homogenizer and rocked for one hour at 4° C., followed by ultracentrifugation for 30 minutes. The cleared lysate was mixed with 10 mL of 50% Ni-NTA (ThermoFisher) resin suspension equilibrated in Buffer A and subjected to batch binding for an hour. After that, the slurry was poured into a glass gravity flow column (Kimble), the flow-through was discarded, and the resin was washed three times with 50 mL of Buffer A supplemented with 0.03% GDN (wash 1) and 1 M NaCl (wash 2) or 20 mM imidazole (wash 3). X1HAS1 was eluted using Elution Buffer (EB) consisting of 25 mM Tris-HCl pH 7.8, 150 mM NaCl, 10% glycerol, 350 mM imidazole, 0.03% GDN in two steps. First, 15 mL of EB was added followed by ~5 min incubation, draining, and addition of another 15 mL of EB and draining. The eluted sample was concentrated using 50 kDa MWCO Amicon centrifugal concentrator (Millipore) to <1 mL for size exclusion chromatography (SEC) using a Superdex200 column (GE healthcare) equilibrated in Gel Filtration Buffer (GFB) consisting of 20 mM Tris-HCl pH 7.8, 150 mM NaCl and 0.02% GDN. The target peak fractions were pooled and concentrated as necessary for subsequent experiments.

### CvHAS Purification

Purification of CvHAS was performed as described (13). All steps were carried out at 4° C. Harvested C43 cells were resuspended in RB (20 mM Tris pH 7.5, 100 mM NaCl, 10% glycerol, 0.5 mM TCEP-HCl), dounce homogenized and mixed with 1 mg/mL egg white lysozyme for 1 hour. Cells were lysed by three passages through a microfluidizer at 18,000 psi, 2 mM PMSF was added after the first passage. Unbroken cells and debris were cleared by centrifugation at 12,500 rpm in a JA-20 rotor. Supernatant from the JA-20 spin was collected to harvest membranes by ultracentrifugation in a Ti-45 rotor at 42,000 rpm for 2 hours. The crude membrane fraction was dounce homogenized in SB (20 mM Tris pH 7.5, 300 mM NaCl, 40 mM imidazole, 10% glycerol, 0.5 mM TCEP, 1% DDM, 0.1% CHS, 1 mM PMSF) and mixed for one hour by gentle inversion. Insoluble material was pelleted by ultracentrifugation at 42,000 RPM in a Ti-45 rotor for 30 minutes. The supernatant was mixed in batch with 5 mL Ni-NTA resin (ThermoFisher), equilibrated in SB, for one hour. Flow-through material was collected by gravity, after which the nickel resin was washed with 20 bed volumes WB1 (20 mM Tris pH 7.5, 1 M NaCl, 10% glycerol, 40 mM imidazole, 0.02% DDM, 0.002% CHS, 0.5 mM TCEP) and 20 volumes WB2 (20 mM Tris pH 7.5, 300 mM NaCl, 10% glycerol, 80 mM imidazole, 0.02% DDM, 0.002% CHS, 0.5 mM TCEP). CvHAS was eluted in 5 volumes EB (20 mM Tris pH 7.5, 300 mM NaCl, 10% glycerol, 320 mM imidazole, 0.02% DDM, 0.002% CHS, 0.5 mM TCEP). The eluate was concentrated and injected on an S200 Increase 10/300 GL column (Cytiva) equilibrated in GFB1 (20 mM Tris pH 7.5, 100 mM NaCl, 0.5 mM TCEP, 0.02% DDM and 0.002% CHS). Fractions containing CvHAS were pooled and concentrated for subsequent reconstitution.

### CvHAS Inverted Membrane Vesicle Preparation

To prepare CvHAS-bearing inverted membrane vesicles (IMVs), 2 L cell pellet was resuspended in Buffer A (20 mM Tris-HCL pH 7.5, 100 mM NaCl, 10% glycerol, 5 mM BME, 2 mM PMSF). Cells were incubated with 1 mg/mL lysozyme for 1 hour before disruption by three passes through a microfluidizer at 18,000 PSI. Unbroken cells were pelleted by centrifugation at 12,500 RPM for 25 minutes in a JA-20 rotor. Cleared lysate was carefully layered onto 40 mL of 2 M sucrose, followed by centrifugation in a Ti-45 rotor (Beckman) at 42,000 rpm for 2 hours. The brown IMV ring was collected, diluted to 60 mL using Buffer B (20 mM Tris-HCl pH 7.5, 100 mM NaCl, 10% glycerol) and subjected to another round of ultracentrifugation for 1 hour. The pelleted IMVs were resuspended in 2 mL of Buffer B, aliquoted, flash-frozen, and stored at −80° C. for subsequent use in activity assays.

### Quantification of HAS Activity by Scintillation Counting

All reagents for biochemical analyses were supplied by Sigma, unless stated otherwise. Protein concentrations in CvHAS IMVs were normalized as described previously (38). Reaction buffer consisted of 40 mM Tris-HCl pH 7.5, 150 mM NaCl, 20 mM MnCl₂, 0.5 mM TCEP, 5 mM UDP-GlcNAc, 5 mM UDP-GlcA and 0.01 μCi/μL [³H]-UDP-GlcNAc. Reactions were carried out at 30° C. for 2 hours. HA digests were performed by adding 2% DDM and 75 U hyaluronidase (MP Biotech) to the reaction and incubating for an additional 10 minutes at 30° C. Reactions were terminated with 3% SDS, and product accumulation was quantified using descending paper chromatography and liquid scintillation counting as previously described (38).

### Electrophoretic HA Size Determination

To assess the size of HA synthesized by X1HAS1, similar reaction conditions were applied to the ones described above. Reaction buffer consisted of 25 mM Tris-HCl pH 7.8, 150 mM NaCl, 0.02% GDN, 20 mM MgCl₂, 5 mM UDP-GlcA and 5 mM UDP-GlcNAc. Reactions were carried out at 37° C. for 3 hours, and at 1 μM HAS concentration. Controls were carried out by digesting the synthesized polymers with hyaluronidase. Synthesis reactions were mixed with SDS-PAGE loading dye and applied to a 1% agarose gel (Ultra-pure agarose, Invitrogen) casted in an Owl B2 system (ThermoFisher). All gels were subjected to electrophoresis at 100 V for 2 hours at room temperature to achieve comparable separation for each run. After the run, the gel was equilibrated in 50% ethanol for 1.5 h and subjected to staining in 0.05% Stains-all (Sigma) in 50% ethanol overnight under light-protection. Post-staining background was reduced by soaking the gel in 20% ethanol for 3-7 days in the dark. The migration of the synthesized HA species was compared to HA standards from Streptococcus equi (Sigma), as well as enzymatically generated ladders: HA LoLadder and HA HiLadder (Hyalose).

HA Extraction from Chlorella Algae

All reagents used for HA extraction were acquired from Sigma-Aldrich unless stated otherwise. 8 mL of PBCV-1 infected NC64A Chlorella algae at 7×10⁸ cells/mL were mixed 1:1 with extraction buffer (2 mM Tris-HCl pH 7.5, 5 mM EDTA, 20 mM NaCl, 0.1% SDS, 0.2 mg/mL DNase, 0.2 mg/mL RNase, 0.2 mg/mL Driselase) by rocking for 1 hour at RT. After 1 hour, Proteinase K was added to a final concentration of 0.1 mg/mL, and the extract was mixed overnight. Algal extract was mixed with 9 volumes of 2:1 CHCl₃:MeOH by vigorous shaking. 3 volumes of CHCl₃ and 3 volumes of MQ-H₂O were added followed by vigorous mixing to complete aqueous extraction. Phase separation was allowed to occur under gravity for 1 hour. The aqueous fraction was removed using a serological pipette and mixed with ice-cold isopropanol at a final concentration of 90%. HA precipitation was allowed to occur on ice for 1 hour. The HA pellet was isolated by centrifugation at 15,000×g for 15 minutes at 4° C. The resulting HA pellet was resuspended in 1 mL of MQ-H₂O and dried to ~50 μL in a speed-vac to concentrate for agarose gel electrophoresis experiments.

### Substrate Turnover Rate Quantification

UDP release during HA synthesis was quantified using an enzyme-coupled assay as previously described (10, 13). Depletion of NADH was monitored at 340 nm every 60 s for 3 hours at 37° C. in a SpectraMax instrument. The raw data was processed in MS Excel and a linear phase of each reaction was determined. The rate of NADH depletion was converted to pmoles of UDP released using a UDP standardized plot (FIG. S1C) for Michaelis-Menten constant determination using GraphPad Prism. For substrate turnover rate quantification of X1HAS1 mutants, pmoles of released UDP were converted to the number of corresponding substrate turnovers per molecule of X1HAS1, per minute. Data is presented relative to wild type activity. All experiments were performed in four to eight replicates and error bars represent standard deviation from the means.

### X1HAS1 Reconstitution and Identification of Specific Fabs

For Fab selection, X1HAS1 was reconstituted into E. coli total lipid nanodiscs (39) using chemically biotinylated MSP1D1. The purified enzyme was mixed with MSP and sodium cholate-solubilized lipids at 40 μM final concentration in 1 mL final volume according to 1:4:80 molar ratio of HAS:MSP:lipids. Detergent removal was initiated 1 hour after mixing all ingredients by adding 200 mg of BioBeads (BioRad) and mixing at 4° C. After an hour, another batch of BioBeads was added, followed by mixing overnight. The following morning (after ~12 hours), the reconstitution mixture was transferred to a fresh tube and the last batch of BioBeads was added, followed by mixing for one hour and SEC using Superdex200 column equilibrated with GFB lacking detergent.

Phage selection was performed as previously described (40, 41). In the first round of selection 400 nM of X1HAS1-loaded nanodiscs diluted in the selection buffer (20 mM HEPES pH 7.5, 150 mM NaCl, 1% BSA) was immobilized on streptavidin paramagnetic beads (Promega). Beads were washed three times in the selection buffer, with 5 mM d-desthiobiotin added during the first wash to block nonspecific binding. Fab phage library E (42) resuspended in selection buffer was added to the beads and incubated for 1 hour with gentle shaking. The beads were washed three times in the selection buffer and then transferred to log-phase E. coli XL1-Blue cells. Phages were amplified overnight in 2×YT medium with ampicillin (100 μg/ml) and M13-KO7 helper phage (109 pfu/mL). Four additional rounds of selection were performed with decreasing target concentration (200 nM, 100 nM, 50 nM, 25 nM) using a KingFisher magnetic beads handler (ThermoFisher). In every subsequent round amplified phage pool from the previous round was used as the input. Prior to being used for selection, each phage pool was precleared by incubation with 100 μl of streptavidin magnetic beads. Additionally, 2 μM of non-biotinylated MSP1D1 nanodiscs were present in the selection buffer to reduce the presence of non-specific binders during rounds two to five. In these rounds selection buffer supplemented with 1% Fos-choline-12 was used to release the target and bound phages from the nanodiscs. Cells infected after the last round were plated on LB agar with ampicillin (100 μg/ml) and phagemids from individual clones were sequenced at the University of Chicago Comprehensive Cancer Center Sequencing Facility to identify unique binders. Single-point phage ELISA was used to validate specificity of unique binders as described previously (41). Fabs were expressed and purified as described (41) and used for activity assays and SEC co-elution experiments with X1HAS1. The strongest non-inhibitory binders were chosen for cryoEM trails and one of those yielded well-structured projections of the HAS-Fab complex.

### CvHAS Nanodisc Reconstitution and Nanobody Complex Formation

Nanobodies were expressed and purified as described earlier (13) and stored in aliquots at −80° C. for further use. Fractions from the initial SEC containing CvHAS were pooled, concentrated and combined with MSP1E3D1 and E. coli Total Lipid Extract (solubilized in 60 mM DDM) at a 1:3:30 ratio. When reconstituting CvHAS in a ternary complex with camelid nanobodies, the initial reconstitution mixture was supplemented with a 3-fold molar excess of appropriate Nb composition (3:3:1 Nb872:Nb881:CvHAS for the GlcNAc primed, UDP-GlcA state and 3:3:1 Nb872:Nb886:CvHAS for disaccharide associated states). The reconstitution was mixed by inversion for 30 minutes before adding BioBeads (BioRad) to about ⅓ of the total volume. After 30 minutes, an additional volume of BioBeads was added, and the reconstitution mixture was allowed to incubate overnight. The next day, another volume of BioBeads was added. After 30 minutes, the mixture was cleared of BioBeads, as well as lipid and protein aggregates by passing through a 0.2 μm cellulose acetate spin filter. The filtered mixture was re-injected on an S200 SEC column equilibrated in GFB2 (20 mM Tris pH 7.5, 100 mM NaCl, 2 mM MnCl₂, 0.5 mM TCEP). CvHAS nanodisc fractions were screened by SDS-PAGE for co-eluting complex components.

X1HAS1 cryoEM Sample Preparation

GDN-solubilized X1HAS1 was used for cryoEM experiments. The purified enzyme was mixed with Fab at a 1:4 molar ratio and incubated overnight at 4° C., followed by SEC using Superose 6 column (Cytiva) equilibrated with GFB2 containing 0.01% GDN.

Initial attempts to prepare cryoEM grids of the purified X1HAS1-Fab complex in the presence of substrates failed to capture a HAS-HA intermediate due to rapid HA accumulation in the sample hampering grid vitrification process. To generate a HA-associated sample, substrates (UDP-GlcA and UDP-GlcNAc, 2.5 mM each, Sigma) and 20 mM MgCl₂ and recombinant HA lyase (0.01 mg/mL, purified as described previously (43)) were included during the overnight Fab incubation, followed SEC. Post-SEC, the sample was concentrated to 8 mg/mL using a 100 kDa MWCO Amicon ultrafiltration membrane. Attempts to generate a GlcNAc-terminated and HA- and UDP-bound sample involved addition of 2.5 mM UDP-GlcNAc and 2.5 mM MgCl₂ after SEC and incubation on ice for 1 hour prior to cryoEM grid vitrification. 4 μL of X1HAS1 sample was applied onto the C-flat 1.2/1.3 grid, glow-discharged for 45 seconds in the presence of 1 drop of amylamine. Grids were blotted for 4 seconds at a blot force of 4, at 4° C. and 100% humidity and plunge-frozen in liquid ethane using Vitrobot Mark IV (FEI). This sample yielded the UDP-bound and gating loop inserted X1HAS1 structure.

CvHAS cryoEM Sample Preparation

For the GlcNAc-primed, UDP-GlcA-bound state of CvHAS, 5 mM GlcNAc was included in the initial nanodisc reconstitution mixture. Purified D302N CvHAS nanodiscs in complex with Nb881 and Nb872 were concentrated to 3.5 mg/mL, then further supplemented with 10 mM MnCl₂, 5 mM GlcNAc, and 5 mM UDP-GlcA, and incubated on ice for 10 minutes. Quantifoil R1.2/1.3 300 mesh grids were glow-discharged for 45 seconds with 1 drop of amylamine. A sample volume of 2.5 μl was applied to each grid at 4° C., 100% humidity, blotted for 12 seconds with a blot force of 4 and plunge frozen in liquid ethane using Vitrobot.

To capture the HA disaccharide bound state, purified CvHAS nanodiscs in complex with Nb881 and Nb872 at 3.0 mg/mL protein concentration were supplemented with 5 mM GlcNAc, 2 mM MnCl₂ and 1 mM UDP-GlcA. GlcNAc extension by UDP-GlcA was allowed to occur on ice for 45 minutes before 3 l of sample was applied to glow-discharged QF R1.2/1.3 grids for plunge freezing.

The sample for CvHAS associated with an HA disaccharide and UDP was obtained by purifying CvHAS nanodiscs in complex with Nb886 and Nb872. The purified complex was concentrated to 7.0 mg/mL and diluted 2-fold with a reaction mixture containing 10 mM UDP-GlcNAc, 10 mM UDP-GlcA and 40 mM MnCl₂. After mixing, the sample was incubated at room temperature for 15 minutes before applying 2.5 μL to glow discharged QF R1.2/1.3 grids for plunge freezing.

### CryoEM Data Collection and Processing

All cryoEM datasets were collected on a Titan Krios equipped with a K3/GIF detector (Gatan) at the Molecular Electron Microscopy Core (University of Virginia School of Medicine). Forty-frame movies were recorded in counting mode at 81,000× nominal magnification, −2.0 to −1.0 μm target defocus, and 50 e-/Å2 total dose.

All datasets were processed in cryoSPARC (44). Raw movies were subjected to patch motion correction and patch contrast transfer function (CTF) estimation. Particles were automatically selected by template picker and sorted by iterative cycles of 2D classification and heterogeneous refinement. To separate HASs bound to their respective ligands, 3D variability and 3D classification approaches were used. The final volumes were refined using non-uniform and local refinements to generate high-resolution maps with 3.0-3.3 Å average resolutions (FIG. S2-s4 and FIG. S7-s8).

### Model Building

To generate the X1HAS1 model, the Alphafold2 (45) prediction was docked into the EM map using Chimera (46) and the model was iteratively real space refined in Coot (47) and Phenix (48). (Table S1).

TABLE S1
CryoEM data collection parameters and model statistics.

| XI-HAS-1 | | | Cv-HAS | | |
|---|---|---|---|---|---|
| apo | HA-bound | UDP-bound | Primed + UDP-GlcA | HA2-bound | HA2 + UDP |

Data collection

| | |
|---|---|
| Microscope | Titan Krios |
| Camera | K3, GIF, 10 eV slit |
| Magnification | 81,000x |
| Voltage (kV) | 300 |
| Dose (e⁻/A²) | 50 |
| Defocus range (μm) | −2.0 to −1.2 |
| Pixel size | 1.08 |

Data processing

| | | | | | | |
|---|---|---|---|---|---|---|
| Symmetry | C1 | C1 | C1 | C1 | C1 | C1 |
| Particles extracted # | 6,543,217 | 11,732,347 | 14,227,871 | 8,797,858 | 12,554,877 | 12,554,877 |
| Final particles # | 305,106 | 171,641 | 76,968 | 176,543 | 115,974 | 220,556 |
| FSC threshold | 0.143 | 0.143 | 0.143 | 0.143 | 0.143 | 0.143 |
| Map sharpening | 98.4 | 94.5 | 85.9 | 133.0 | 109.1 | 108.6 |

Model refinement

| | | | | | | |
|---|---|---|---|---|---|---|
| Initial model used | AF2 | XI-HAS-1 apo | XI-HAS-1 apo | Cv-HAS Apo | Cv-HAS Apo | Cv-HAS Apo |
| Model resolution | 3.2 | 3.2 | 3.4 | 3.2 | 3.2 | 3.0 |
| Non-hydrogen atoms | 5993 | 6112 | 6138 | 6089 | 6080 | 6086 |
| Protein residues | 746 | 746 | 762 | 742 | 746 | 743 |
| Ligands | 0 | 9 | 2 | 6 | 6 | 6 |

B factors

| | | | | | | |
|---|---|---|---|---|---|---|
| Protein | 60.04 | 60.68 | 50.09 | 71.02 | 106.71 | 58.89 |
| Ligand | — | 76.67 | 60.44 | 79.90 | 111.91 | 57.39 |

Rms deviations

| | | | | | | |
|---|---|---|---|---|---|---|
| Bond lengths | 0.002 | 0.002 | 0.002 | 0.010 | 0.005 | 0.004 |
| Bond angles | 0.461 | 0.462 | 0.505 | 0.935 | 0.997 | 1.024 |

Validation

| | | | | | | |
|---|---|---|---|---|---|---|
| MolProbity score | 1.56 | 1.57 | 1.57 | 1.20 | 1.66 | 1.52 |
| Clashscore | 5.38 | 6.12 | 6.24 | 3.16 | 6.29 | 4.38 |
| Rotamer outliers (%) | 0.00 | 0.00 | 0.00 | 0.16 | 1.25 | 0.00 |

Ramachandran plot

| | | | | | | |
|---|---|---|---|---|---|---|
| Favored | 96.06 | 96.47 | 96.55 | 97.54 | 96.34 | 95.65 |
| Allowed | 3.94 | 3.53 | 3.45 | 2.46 | 3.66 | 4.22 |
| Outliers | 0.00 | 0.00 | 0.00 | 0.00 | 0.00 | 0.14 |

Across all models, most of X1HAS1 residues were able to be modeled with the exception of the flexible loop of the GT domain (residues 172-193), the N-terminal (residues 1-14) and C-terminal (residues 569-588) extensions.

Substrate- and HA oligo-bound CvHAS models were generated by docking in the proper ligands in Coot using the previously published CvHAS model (PDB ID 7SP6), followed by real-space refinement in Phenix. For the HA2 and UDP bound structure of CvHAS, coordinates for Nb886 were first generated by Alphafold2. Nb886 coordinates were docked into the cryoEM density map and merged with the CvHAS-Nb872 coordinates. The merged complex and ligand were iteratively real-space refined.

All model figures were prepared using PyMOL (49) or ChimeraX (50).

### Results

### X1HAS1 Synthesizes HA Polymers in a Megadalton Size Range

X1-HAS-1 was expressed in Spodoptera frugiperda (Sf9) cells and purified using immobilized metal affinity (IMAC) and size-exclusion chromatography (SEC) in glyco-diosgenin (GDN) detergent (see Materials and Methods). Based on SEC and cryoEM analyses (see below), the catalytically active enzyme is monomeric (FIG. 1C).

HAS transfers the UDP-activated glycosyl unit (the donor sugar) to the non-reducing end of the nascent HA polymer (the acceptor sugar). This reaction generates UDP and elongated HA as reaction products (FIG. 1A). Catalytic activity can be assessed by monitoring the release of UDP in an enzyme-coupled reaction, as previously described (10, 13), or quantifying HA, either radiometrically or by dye staining following electrophoresis.

X1HAS1 exhibits robust catalytic activity in the presence of magnesium cations, whereas its activity is reduced to about 30% in the presence of magnesium cations, whereas its activity is reduced by about 30% when manganese is used instead as the cofactor. The synthesized polysaccharide is readily degraded by hyaluronidase, demonstrating that it is indeed authentic HA (Extended Data FIGS. 1A and B). Agarose gel electrophoresis of the in vitro synthesized HA followed by Stains-All staining reveals increasing polymer lengths over a ~120 min synthesis reaction before stalling (FIG. 1D). Notably, at completion, X1HAS1 synthesizes HA polymers of a consistent length that migrate above a 1.6 MDa HA marker. This material is comparable to HA in vitro synthesized by Streptococcus equisimilis (Se) HAS. In contrast, CvHAS produces polydisperse low molecular weight HA of about 30 to 200 200 kDa (FIG. 1E).

Kinetic analyses were performed by titrating one substrate at a saturating concentration of the other and real-time quantification of the released UDP product (FIG. 1F and Extended Data FIG. 1C). The results reveal Michaelis-Menten constants for UDP-GlcNAc and UDP-GlcA of about 470 and 370 μM, respectively, and an overall catalytic rate of approximately 30 substrate turnovers per minute. Similar biosynthesis rates have recently been reported for cellulose synthase (14). Monitoring the length distribution of the synthesized HA under these substrate conditions (one limiting, the other in excess) shows increasing HA lengths up to a substrate concentration of 2.5 mM (FIG. 1G). Based on the observed HA product lengths (~1.6 MDa produced within 90 min, FIG. 1D), the electrophoretic analysis suggests a catalytic rate on the same order of magnitude as determined by UDP quantification, yet about 2-times higher, for unknown reasons.

In vivo, HA biosynthesis could stall due to substrate depletion, competitive UDP inhibition, or HA accumulation. To analyze the impact of sustained high substrate concentrations during HA biosynthesis, an in vitro synthesis reaction was supplemented with both substrates two, four, and six hours after initiation, while converting the accumulating UDP to non-inhibitory UTP (FIG. 1H and Extended Data FIG. 1D-F). These conditions further extend the synthesized HA polymers to products substantially exceeding the 1.6 MDa marker. Extension is only observed when both substrates are supplied in excess (2.5 mM). If one substrate (UDP-GlcNAc) remains at a limiting concentration (0.1-1 mM), no increase in HA size is observed, likely due to premature HA release, followed by re-initiation.

### Architecture of X1-HAS-1 in a Resting State

A non-inhibitory antibody Fab fragment was selected to facilitate cryoEM structural analyses of X1HAS1 (FIGS. 2A and B and Extended Data FIG. 2). The purified X1HAS1-Fab complex was either used directly for cryoEM grid preparation or incubated with substrates to obtain snapshots of biosynthetic intermediates (Extended Data FIGS. 2-4 and Extended Data Table 1).

Extended Data Table 1. CryoEM data collection, refinement, and validation statistics.

| | | | | Cv-HAS | | Cv-HAS |
|---|---|---|---|---|---|---|
| | XI-HAS-1 | XI-HAS-1 | XI-HAS-1 | primed | Cv-HAS | HA2 + |
| | Apo | HA-bound | UDP-bound | UDP-GlcA-bound | HA2-bound | UDP-bound |
| | (EMDB-40591) | (EMDB-40594) | (EMDB-40598) | (EMDB-40623) | (EMDB-40622) | (EMDB-40624) |
| | (PDB 88MM) | (PDB 88MN) | (PDB 88MP) | (PDB 88ND) | (PDB 88NC) | (PDB 88NE) |
| Data collection and processing | | | | | | |
| Magnification | 81,000 | 81,000 | 81,000 | 81,000 | 81,000 | 81,000 |
| Voltage (kV) | 300 | 300 | 300 | 300 | 300 | 300 |
| Electron exposure (e−/Å²) | 50 | 50 | 50 | 50 | 50 | 50 |
| Defocus range (μm) | −2.0 to 1.2 | −2.0 to 1.2 | −2.0 to 1.2 | −2.0 to 1.2 | −2.0 to 1.2 | −2.0 to 1.2 |
| Pixel size (Å) | 1.08 | 1.08 | 1.08 | 1.08 | 1.08 | 1.08 |
| Symmetry imposed | C1 | C1 | C1 | C1 | C1 | C1 |
| Initial particle images (no.) | 6,543,217 | 22,061,285 | 26,290,833 | 7,978,429 | 3,663,447 | 12,554,677 |
| Final particle images (no.) | 337,002 | 169,894 | 136,942 | 176,543 | 56,954 | 220,556 |
| Map resolution (Å) | 3.2 | 3.0 | 3.2 | 3.2 | 3.3 | 3.0 |
| FSC threshold | 0.143 | 0.143 | 0.143 | 0.143 | 0.143 | 0.143 |
| Map resolution range (Å) | 2.7-47.6 | 2.6-45.1 | 2.8-49.0 | 2.4-10.1 | 2.9-51.5 | 2.3-8.7 |
| Refinement | | | | | | |
| Initial model used (PDB code) | Alphafold2 | 88MM | 88MM | 78P6 | 78P6 | 78P6 |
| Model resolution (Å) | 3.2 | 3.0 | 3.2 | 3.2 | 3.3 | 3.0 |
| FSC threshold | 0.143 | 0.143 | 0.143 | 0.143 | 0.143 | 0.143 |
| Map sharpening B factor (Å²) | 95.4 | 102.0 | 87.0 | 133.0 | 117.7 | 108.6 |
| Model composition | | | | | | |
| Non-hydrogen atoms | 5930 | 6138 | 6097 | 6090 | 5928 | 6025 |
| Protein residues | 738 | 749 | 757 | 742 | 728 | 735 |
| Ligands | 0 | 9 | 2 | 6 | 6 | 6 |
| B factors (Å²) | | | | | | |
| Protein | 75.6 | 35.98 | 73.11 | 68.63 | 58.44 | 52.18 |
| Ligand | — | 50.07 | 60.99 | 82.53 | 54.72 | 55.03 |
| R.m.s. deviations | | | | | | |
| Bond lengths (Å) | 0.004 | 0.002 | 0.002 | 0.005 | 0.004 | 0.008 |
| Bond angles (°) | 0.660 | 0.519 | 0.526 | 0.985 | 0.859 | 1.054 |
| Validation | | | | | | |
| MolProbity score | 1.63 | 1.48 | 1.64 | 1.20 | 1.58 | 1.54 |
| Clashscore | 6.97 | 4.61 | 6.29 | 4.23 | 5.36 | 4.92 |
| Poor rotamers (%) | 0.31 | 0.15 | 0.3 | 0.0 | 0.32 | 0.16 |
| Ramachandran plot | | | | | | |
| Favored (%) | 96.28 | 96.35 | 95.72 | 98.22 | 95.81 | 95.86 |
| Allowed (%) | 3.72 | 3.65 | 4.15 | 1.64 | 4.19 | 4.00 |
| Disallowed (%) | 0.00 | 0.00 | 0.13 | 0.14 | 0.00 | 0.14 |

X1HAS1's cytosolic GT domain interacts with two N-terminal and four C-terminal transmembrane helices (TMHs) as well as three cytosolic interface helices (IFH1-3) (FIGS. 2B-E). The IFHs surround the entrance to curved channel that ends with a lateral portal halfway across the membrane, formed by IFH3 and TMHs 5 and 6 (FIG. 2D). The function of the curved channel/pathway is unknown as it is not used for HA translocation, except for its cytosolic entrance (see below). IFH2 contains the conserved QxxRW motif (Extended Data FIG. 5) of which Trp382 is pivotal for positioning the acceptor sugar right at the entrance to the TM channel.

The X1HAS1 architecture is consistent with an AlphaFold predicted structure of human HAS1 and HAS2 and the previously determined CvHAS structure (Extended Data FIG. 6A-6D; 13), with the exception that TMH1 is clearly resolved in the X1HAS1 maps (FIG. 2C and Extended Data FIG. 2C). This curved helix rests against the cytosolic connection of IFH2 and TMH3 (FIG. 2E).

Vertebrate HASs contain a conserved WGTSGRR/K motif in a cytosolic ‘gating loop’ connecting IFH3 with TMH5. A similar motif in a loop close to the active site is found in chitin and cellulose synthases (13, 17-19 and 20) (Extended Data FIG. 4D-4F). Although the gating loop is unresolved in the apo X1HAS1 structure, its flanking residues Thr485 and Tyr503 at the C-terminus of IFH3 and beginning of TMH5, respectively, position it right above the catalytic pocket (FIG. 2E) where it could facilitate substrate binding (11). In analogy to cellulose synthase and functional analyses described below, this loop is referred to as X1HAS1's ‘gating loop’.

### HAS Coordinates a Nascent HA Polymer Inside an Electropositive Channel

To stabilize an HA-associated translocation intermediate, an in vitro HA synthesis reaction was performed in the presence of hyaluronan lyase to trim polymers emerging from HAS' TM channel. CryoEM maps of HA-bound X1HAS1 reveal continuous non-proteinaceous density running from the catalytic pocket to its extracellular surface through an electropositive channel (FIG. 3A, and Extended Data FIG. 3C). The density accommodates nine glycosyl units, denoted 1-9 starting at the non-reducing end at the acceptor position. The assignment of the HA register is based on the following observations: 1) The shape of the non-reducing end terminal glycosyl unit resembles a GlcNAc moiety and is consistent with maps of GlcNAc primed CvHAS (Extended Data FIG. 3C). 2) Following this register, the densities at the polymer's third and fifth glycosyl positions are also consistent with GlcNAc moieties, as expected for an alternating GlcNAc-GlcA repeat unit. This register is also consistent with previous analyses of CvHAS demonstrating stable coordination of GlcNAc, but not GlcA, at the acceptor position (13). 3) The carboxylate substituents of the GlcA units are expected to be weak or even absent in the electron potential maps (Extended Data FIG. 3C) Despite confidence in the register of the modeled HA chain, the exact orientation of the glycosyl units is less certain. Disaccharide repeat units of the HA polymer likely enter the translocation channel in two orientations with their acetamido and carboxylate groups pointing roughly in opposite directions (see below). Thus, in addition to the modeled conformation, contributions from an alternatively oriented polymer to the observed cryoEM map cannot be exclude.

The first GlcNAc (GlcNAc-1) sits inside a collar of invariant residues, including Tyr288, Phe292, Cys307, Trp382, Ser385, as well as Asp342 and Arg343 of the conserved GDDR motif (FIG. 3A and Extended Data FIG. 5). Following the acceptor site, the conserved Arg296 and Arg287 of IFH1 are in close proximity to GlcA-2 and likely stabilize GlcA units inside the channel. Past this point, the channel dimensions widen and HA's interactions with side chains are less extensive.

While HA's first three glycosyl units enter the channel in a co-planar conformation, the polymer rotates by about 900 around the GlcNAc-3-GlcA-4 glycosidic bond (FIG. 3A and Extended Data FIG. 3C). The rotation is evident from the planes of the resolved glycopyranose rings. It occurs at a central widening of the TM channel that can accommodate spontaneous structural rearrangements of the polysaccharide, as observed in solution (21). At this widening, His72 and Lys448 contribute to the channel's electropositive character (FIG. 3A).

The next disaccharide unit (GlcA-6 and GlcNAc-7) is rotated by about 450 relative to the preceding glycosyl units and the following disaccharide (GlcA-8 and GlcNAc-9) exhibits a similar rotation relative to the preceding pair (Extended Data FIG. 3C). Past GlcNAc-9, the discontinuous EM map prevents further interpretations.

Of note, about halfway across the membrane, the HA polymer is encircled by a Met-rich ring of hydrophobic residues, including Met69, Phe414, Ile418, Ile441, Met444, and Met472 (FIG. 3A). Past this hydrophobic ring, the nascent HA chain is surrounded by moderately conserved hydrophilic and hydrophobic residues, including Tyr46, Glu49, Gln51, Ser61, Thr421, Leu425, Asn433, Trp436, and Cys440 (FIG. 3A). This channel segment is strikingly devoid of positively charged residues, unlike the preceding section.

Bending of TMHs opens the HA translocation channel HA translocation requires conformational changes of X1HAS1 to create a continuous TM channel. Compared to the resting conformation described above, the N-terminal half of TMH2 near the extracellular water-lipid interface bends away from TMH4 (FIG. 3B). Bending occurs around a conserved GLYG motif (residues 63 to 66) that places two Gly residues at the interface with TMH4. Furthermore, the extracellular helical turns of TMH1 and TMH2 to unwind to form a short β-hairpin with residues of the TMH1-2 loop (FIG. 3B). Although only the loop's backbone is resolved in X1HAS1's apo conformation (Extended Data FIG. 2C), it is evident that the loop lids the extracellular channel exit in the absence of HA. Upon channel opening, the loop flips towards the membrane and rotates by approximately 900 to run roughly parallel to the membrane surface. Additionally, the N-terminal half of TMH6 bends by approximately 10° towards TMH2, around the conserved Gly547 (FIG. 3B). In the new position, His543 is in hydrogen bonding distance to the backbone carbonyl oxygen of Ile58. Compared to the open conformation of CvHAS observed in the presence of a GlcNAc primer (13), the unwinding of the N-terminal segment of TMH2 in X1HAS1 creates a larger extracellular channel portal. Further, the bending of TMH6 is not observed in the HA-free open channel conformation of CvHAS (Extended Data FIG. 6D).

HAS' Gating Loop Inserts into the Catalytic Pocket

Attempts to stabilize a HA translocation intermediate also resulted in an HA-free but UDP-bound conformation (FIG. 3C and Extended Data FIG. 4). In this structure, UDP is coordinated via conserved motifs of the GT domain, as previously described for CvHAS and other processive and non-processive GTs of the GT-A fold (13, 17, 18, 22-24). The map resolves X1HAS1's gating loop, connecting IFH3 with TMH5 (residues 485 to 503) and containing the conserved WGTSGRK/R sequence (residues 491 to 497) (Extended Data FIG. 4, 5). The loop inserts into the catalytic pocket and interacts with the nucleotide (FIG. 3C). In this conformation, Trp491 forms a cation-π interaction with Arg381 of the QxxRW motif in IFH2, which in turn forms a salt bridge with the nucleotide's diphosphate group. The indole ring of Trp491 runs approximately perpendicular to the uracil moiety, placing its Ne within hydrogen bonding distance to UDP's α-phosphate. This phosphate group is also contacted by the side chain of the following Thr493. Arg496, the penultimate residue of the WGTSGRK/R motif inserts into a negatively charged pocket formed by Glu109, Asp242, Glu367 and UDP's α-phosphate (FIG. 3C).

### The Gating Loop is Necessary for HA Biosynthesis

Site-directed mutagenesis was used to reveal the gating loop's functional importance in two model systems, X1HAS1 as well as CvHAS (FIG. 4). For X1HAS1, replacing Trp491 of the WGTSGRK/R motif with Ala (W491A) abolishes HA biosynthesis during a 60-min synthesis reaction (denoted ‘short reaction’), while its substitution with Phe (W491F) results in a lower molecular weight product (FIG. 4A, B). Similarly, quantifying UDP release by these mutants shows abolished and reduced substrate turnover rates, respectively, compared to the wild type (WT) enzyme (FIG. 4C). Interestingly, over an 8 h synthesis reaction (‘long reaction’), the W491F mutant can produce HA polymers similar in size to the WT enzyme. Likewise, replacing the following Thr493 (WGTSGRK/R) with Ala or Ser reduces the catalytic rate to about 20-25% of that of the WT enzyme (FIG. 4C). Strikingly, the T493A and T493S mutants generate HA exceeding the size of the WT product even during a ‘short’ synthesis reaction (FIG. 4B). The size differences are more pronounced on a lower percentage agarose gel (Extended Data FIG. 6E). Further, replacing Arg496 (WGTSGRK/R) with Ala abolishes catalytic activity while an R496K mutant produces polymers of slightly increased length and greater polydispersity in short and long synthesis reactions, respectively (FIG. 4B).

Similar results were obtained for CvHAS by quantifying HA using scintillation counting (FIG. 4D). Here, the gating loop contains a WGTRG sequence. Replacing the motif's Trp residue (Trp454) with Ala or Phe is incompatible with function and so is the substitution of the following Gly455 with Ala (WGTRG). Replacing the Arg residue (Arg457) with Lys reduces CvHAS's activity by ~60% relative to WT.

### HA Coordination Modulates Polymer Length

Processive HA biosynthesis requires sustained interactions of HAS with HA between elongation steps. To test how HA coordination affects the HA length distribution, active site and channel-lining residues were altered and HA size and UDP release were monitored.

X1HAS1's active site contains two conserved cysteines. Cys307 belongs to the ‘switch loop’ at the back of the active site, adjacent to the GlcNAc acceptor (FIGS. 3C and 4A) where it coordinates the acceptor sugar. Replacing Cys307 with Ala or Ser leads to the production of low amounts of polydisperse HA (FIG. 4B). The second Cys, Cys337, is part of a flexible ‘priming loop’ located to one side of the catalytic pocket (FIGS. 3C and 4A). This residue can be replaced with Ala or Ser, resulting in HA products similar to wild type, with a slight length reduction for the C337S mutant (FIG. 4B).

Also at the active site, replacing the conserved Lys218 with Ala or Arg abolishes substrate turnover and HA synthesis (FIGS. 4E and F). Similarly, Arg381 of the QxxRW motif, located in IFH2 right above the catalytic pocket, cannot be replaced with either Ala or Lys. This residue interacts with the nucleotide's pyrophosphate group in UDP- and substrate-bound states (see below (25)).

Inside the TM channel, the polymer's GlcA-2 is near Arg287 and Arg296 of IFH1 (FIGS. 3A and 4A). Substituting these residues with Ala reduces substrate turnover rates to about 15 and 30%, respectively, compared to WT, resulting in the generation of short HA oligosaccharide species (FIG. 4E, F). Replacing the residues with Lys, however, results in HAS mutants with similar or even increased substrate turnover rates (compared to WT) that produce polydisperse (R287K) and low molecular weight (R296K) HA products in short and long synthesis reactions (FIG. 4E, F).

Farther inside the channel, His72 and Lys448 are adjacent to GlcA-4. Compared to the WT enzyme, the H72A, K448A and K448R mutants display between ~50-75% and the H72F substitution about 10% of catalytic activity (FIG. 4E). In short synthesis reactions, all mutants produce HA polymers of reduced length, with the H72A product being closest to WT (FIG. 4F). In a long synthesis reaction, however, the H72F and K448R variants produce polymers equivalent to or exceeding the HA length obtained from the WT enzyme (FIG. 4F and Extended Data FIG. 6D). The observed differences in electrophoretic mobility suggest a HA size difference of up to 0.5 MDa for some mutants, including T493S and K448R (FIG. 4B, F and Extended Data FIG. 6E).

Attempts to modulate the product size distribution of CvHAS were unsuccessful. Isolating natively produced HA from infected Chlorella algae confirmed the production of low molecular weight HA in vivo (FIG. 4G and Extended Data FIG. 6F, G). Comparing CvHAS polymers produced in a detergent micelle or liposome environment (using E. coli total lipid extract) identifies a moderate increase in HA length when produced from proteoliposomes or in a GDN micelle, compared to the native product. The longer CvHAS-produced polymers are comparable to products obtained from the X1HAS1 R287A, R296A and R296K mutants (FIG. 4F, G and Extended Data FIG. 6G).

### Substrate Binding Repositions the GlcNAc Primer

Upon priming with a GlcNAc monosaccharide (13), HAS attaches GlcA to the C3 hydroxyl group of the primer to form the GlcA-GlcNAc disaccharide repeat unit. To gain insights into this step, the high-quality cryoEM maps routinely obtained for Cv-HAS were utilized, facilitated by two camelid nanobodies (13). The catalytically inactive CvHAS D302N mutant (13, 26) in complex with two nanobodies was incubated with GlcNAc and Mn²⁺.UDP-GlcA prior to grid preparation (Extended Data FIG. 7 and Table 1). In the resulting cryoEM map, the GlcNAc primer is well resolved and occupies the same acceptor position as previously reported, stacking against Trp342 (FIGS. 5A and B, Extended Data FIG. 7) (13). In the presence of the UDP-GlcA substrate, however, the primer rotates by approximately 600 placing its acetamido group away from the active site (FIG. 5E, F). This rotation positions the primer's C3 hydroxyl group closer to the putative base catalyst Asp302 (replaced with Asn in this construct). In this position, the acetamido group's carbonyl oxygen is in proximity to the backbone amide nitrogen of Gly270. The sugar's ring oxygen is adjacent to the sulfhydryl group of the conserved Cys267, its C1 hydroxyl may form a water-mediated interaction with Arg256, and the C6 hydroxyl group is in proximity to Ser345. All acceptor-surrounding residues are conserved.

CvHAS binds both of its substrates in similar binding poses, with UDP being coordinated by two manganese ions, as previously described (13) (FIG. 5E, F). The donor GlcA sugar sits roughly underneath Trp342 of the QxxRB motif. Its C6 carboxylate is incompletely resolved, as often observed in electron potential maps. In this binding pose, however, the carboxylate occupies a pocket formed by the C-terminal end of the priming loop (residues 298 to 300), the following helix that begins with the invariant GDDR motif (residues 300-303), as well as the priming GlcNAc sugar (FIG. 5E). Further, Arg341 of the QxxRW motif is in close proximity to the donor's carboxylate (FIG. 5F). Additional residues surrounding the donor sugar include Asp201, Lys177, and the introduced Asn302 (D302N). The distance between the acceptor's C3 hydroxyl group and the donor's C1 carbon atom is long (about 5.2 Å), indicating that additional conformational changes are necessary for glycosyl transfer.

Insights into Stepwise HA Elongation

Binding of UDP-GlcA to GlcNAc-primed HAS generates a HA disaccharide with GlcA at the non-reducing end. This step was visualized in two experiments. First, exposure of WT CvHAS to both of its substrates resulted in a UDP and HA disaccharide bound conformation, interpreted as a state directly after glycosyl transfer and prior to UDP release (FIG. 5C and Extended Data FIG. 8 and Table 1). The density at the acceptor site is consistent with GlcNAc, preceded by weaker density inside the catalytic pocket, assigned to GlcA. Because the UDP and HA disaccharide densities are not clearly separated, we cannot exclude the presence of an overlapping minor second state in which CvHAS is bound to UDP-GlcA and a GlcNAc monosaccharide primer (released from UDP-GlcNAc).

This situation was improved in a second experiment. CvHAS was primed with a GlcNAc monosaccharide and then incubated with UDP-GlcA for elongation with GlcA. The obtained cryoEM map resolves a GlcNAc at the acceptor position that is elongated by a single sugar unit extending into the catalytic pocket (FIG. 5D and Extended Data FIG. 9 and Table 1). No UDP density is observed at the active site; thus, this pose likely represents a state post glycosyl transfer and UDP release. Although clearly attached to the GlcNAc moiety, the extending GlcA sugar is flexible due to sparse coordination inside the catalytic pocket. We note that the carboxylate group of GlcA, either when extending the priming GlcNAc unit or as part of the donor sugar, likely occupies the same pocket of the catalytic site.

### DISCUSSION

HA, cellulose and chitin synthases are multitasking enzymes that secrete high molecular weight polysaccharides. Mechanisms by which the enzymes control product lengths and thus the polymers' physical properties are largely unresolved. The narrow size distribution of the X1HAS1-synthesized HA underscores high processivity of biosynthesis. X1HAS1's product size distribution broadens significantly during prolonged in vitro synthesis reactions, perhaps due to substrate depletion and/or product inhibition.

Processive HA biosynthesis requires a stable HA-HAS association. Accordingly, modulating HA coordination inside the secretion channel profoundly affects the HA length distribution. Two Arg-to-Lys substitutions within X1HAS1's IFH1 (R287K and R296K) abolish length control or lead to early termination of HA biosynthesis. These residues at the entrance to the TM channel are likely needed for stabilizing the nascent chain for elongation. Additionally, replacing K448, about halfway across the channel, with Ala or Arg substantially reduces the enzyme's catalytic rate, leading to shorter yet discrete product lengths. Notably, mutations reducing X1HAS1's catalytic rate while not changing its processivity give rise to higher molecular weight HA polymers. In vivo, similar effects may be achieved by limiting substrate availability and/or post-translational modifications of the enzyme (27, 28). Thus, differences in HA length associated with HAS isoforms could result from different metabolic states of the expressing tissues.

Collectively, the observed product size distributions synthesized by the ensemble of X1HAS1 mutants reflect the HA size range produced by vertebrate HAS isoenzymes (2, 7). Although the residues analyzed in this study are conserved across HAS isoforms, disparities in channel dynamics and thus HA coordination may differ between isoforms. Thus, physiological differences in HA size likely arise from variations in substrate availability, HA coordination, and/or metabolic states of the expressing cells and tissues, as previously suggested (29).

Among the HASs studied biochemically and structurally so far, CvHAS' TM architecture is likely most flexible, due to a disordered N-terminal TM segment (13). Accordingly, CvHAS exhibits the lowest processivity in vitro and in vivo, presumably due to weak HA coordination during translocation. Modulation of its product size by different membrane mimetics supports this interpretation. Recent analyses of HA biosynthesis in subterranean mammals pinpointed HAS expression levels and hyaluronidase activities as important determinants of HA size (30).

The shape and electropositive character of X1HAS1's TM channel contrast the flat, acidic channel formed by cellulose synthase (22, 31). Because the channel's gate is near the extracellular water-lipid interface, channel opening is estimated to be induced by HA polymers exceeding 3-4 glycosyl units (FIG. 6). In this case, the nascent chain and the channel's central hydrophobic ring likely prevent water flux across the membrane, similar to other polysaccharide secretion systems (32). The observed rotation of the nascent HA chain inside the secretion channel may generate energetically favorable conformations contributing to HA translocation and/or preventing backsliding.

HAS' gating loop resembles the corresponding loops in cellulose and chitin synthases (20). While the precise function of the loop is unclear, site directed mutagenesis experiments of HA and cellulose synthases demonstrate its profound importance for catalytic activity (20). Controlling its flexibility could be a regulatory mechanism, similar to bacterial cellulose synthase (19).

Our new viral HAS structures demonstrate that, upon extending a GlcNAc primer with GlcA, the newly added sugar protrudes into the catalytic pocket, without spontaneous translocation into the secretion channel. In this position, the terminal GlcA would overlap with the sugar moiety of the next donor substrate. Therefore, it is proposed that binding of a new substrate molecule translocated the GlcA-extended nascent chain into the channel to avoid steric clashes (FIG. 6). The new substrate molecule (UDP-GlcNAc) may transiently stabilize GlcA at the acceptor position.

Extending a GlcA acceptor with GlcNAc would generate a HA polymer with GlcA at the acceptor position. Previous MD simulations revealed that this register is unstable, leading to spontaneous HA translocation to place the added GlcNAc moiety at the acceptor site (FIG. 6) (13). Such a register-dependent spontaneous HA translocation is consistent with the experimental observations that GlcNAc terminated polymers end at the acceptor site and that this binding pose is not changed after elongation with a GlcA unit (FIG. 6).

Considering the narrow entry into the HA secretion channel, HA must enter the channel in a flat, ribbon-like conformation. This is only possible if newly added glycosyl units can rotate around the glycosidic linkage within the active site to adopt a favorable conformation (FIG. 6). A similar rearrangement has been suggested for terminal glucosyl units in cellulose synthase (33, 34). The register-dependent rearrangements would result in different orientations of the HA disaccharide units within the polymer (FIG. 6). Accordingly, the experimental HA density may reflect an average of two HA orientations. The channel dimensions widen sufficiently past the first three glycosyl units to accommodate further structural rearrangements of HA. Taken together, the structural and functional analyses provide a molecular framework for engineering polysaccharide biosynthesis systems for a plethora of biomedical, agricultural, and tissue engineering purposes.

### BIBLIOGRAPHY

1 Vigetti, D. et al. Hyaluronan: biosynthesis and signaling. Biochim Biophys Acta 1840, 2452-2459, (2014).
2 Cowman, M. K., Lee, H.-G., Schwertfeger, K. L., McCarthy, J. B. & Turley, E. A. The Content and Size of Hyaluronan in Biological Fluids and Tissues. Front Immunol 6, 261, (2015).
3 Cyphert, J. M., Trempus, C. S. & Garantziotis, S. Size Matters: Molecular Weight Specificity of Hyaluronan Effects in Cell Biology. Int J Cell Biol 2015, 563818, (2015).
4 Band, P. A. et al. Hyaluronan molecular weight distribution is associated with the risk of knee osteoarthritis progression. Osteoarthr Cartilage 23, 70-76, (2015).
5 Petrey, A. C. & de la Motte, C. A. Hyaluronan, a crucial regulator of inflammation. Front Immunol 5, 101, (2014).
6 Roughley, P., Lamplugh, L., Lee, E., Matsumoto, K. & Yamaguchi, Y. The role of hyaluronan produced by Has2 gene expression in development of the spine. Spine 36, E914-920, (2011).
7 Itano, N. et al. Three isoforms of mammalian hyaluronan synthases have distinct enzymatic properties. J Biol Chem 274, 25085-25092, (1999).
8 Rapport, M. M., Weissmann, B., Linker, A. & Meyer, K. Isolation of a crystalline disaccharide, hyalobiuronic acid, from hyaluronic acid. Nature 168, 996-997, (1951).
9 Lombard, V., Ramulu, H. G., Drula, E., Coutinho, P. M. & Henrissat, B. The carbohydrate-active enzymes database (CAZy) in 2013. Nucleic Acids Res 42, D490-D495, (2014).
10 Hubbard, C., McNamara, J., Azumaya, C., Patel, M. & Zimmer, J. The hyaluronan synthase catalyzes the synthesis and membrane translocation of hyaluronan. J Mol Biol 418, 21-31, (2012).
11 Bi, Y., Hubbard, C., Purushotham, P. & Zimmer, J. Insights into the structure and function of membrane-integrated processive glycosyltransferases. Curr Opin Struct Biol 34, 78-86, (2015).
12 DeAngelis, P., Jing, W., Graves, M., Burbank, D. & Van Etten, J. Hyaluronan synthase of chlorella virus PBCV-1. Science 278, 1800-1803, (1997).
13 Maloney, F. P. et al. Structure, substrate recognition and initiation of hyaluronan synthase. Nature 604, 195-201, (2022).
14 DeAngelis, P. L. & Achyuthan, A. M. Yeast-derived recombinant DG42 protein of Xenopus can synthesize hyaluronan in vitro. J Biol Chem 271, 23657-23660, (1996).
15 Pummill, P. E., Achyuthan, A. M. & DeAngelis, P. L. Enzymological characterization of recombinant Xenopus DG42, a vertebrate hyaluronan synthase. J Biol Chem 273, 4976-4981, (1998).
16 DeAngelis, P. L. & Zimmer, J. Hyaluronan synthases; mechanisms, myths, & mysteries of three types of unique bifunctional glycosyltransferases. Glycobiol, (2023).
17 Ren, Z. et al. Structural basis for inhibition and regulation of a chitin synthase from Candida albicans. Nat Struct Mol Biol 29, 653-664, (2022).
18 Chen, W. et al. Structural basis for directional chitin biosynthesis. Nature 610, 402-408, (2022).
19 Morgan, J. L. W., McNamara, J. T. & Zimmer, J. Mechanism of activation of bacterial cellulose synthase by cyclic di-GMP. Nature Struct Mol Biol 21, 489-496, (2014).
20 Verma, P., Kwansa, A. L., Ho, R. Y., Yingling, Y. G. & Zimmer, J. Insights into substrate coordination and glycosyl transfer of poplar cellulose synthase-8. Structure 31, 1166-+, (2023).
21 Almond, A., DeAngelis, P. L. & Blundell, C. D. Hyaluronan: the local solution conformation determined by NMR and computer modeling is close to a contracted left-handed 4-fold helix. J Mol Biol 358, 1256-1269, (2006).
22 Morgan, J., Strumillo, J. & Zimmer, J. Crystallographic snapshot of cellulose synthesis and membrane translocation. Nature 493, 181-186, (2013).
23 Charnock, S. J. & Davies, G. J. Structure of the nucleotide-diphospho-sugar transferase, SpsA from Bacillus subtilis, in native and nucleotide-complexed forms. Biochem 38, 6380-6385, (1999).
24 Taujale, R. et al. Deep evolutionary analysis reveals the design principles of fold A glycosyltransferases. Elife 9, (2020).
25 Morgan, J. L. et al. Observing cellulose biosynthesis and membrane translocation in crystallo. Nature 531, 329-334, (2016).
26 Blackburn, M. R. et al. Distinct reaction mechanisms for hyaluronan biosynthesis in different kingdoms of life. Glycobiol 28, 108-121, (2018).
27 Goentzel, B. J., Weigel, P. H. & Steinberg, R. A. Recombinant human hyaluronan synthase 3 is phosphorylated in mammalian cells. Biochem J 396, 347-354, (2006).
28 Vigetti, D. & Passi, A. Hyaluronan synthases posttranslational regulation in cancer. Adv Cancer Res 123, 95-119, (2014).
29 Vigetti, D., Viola, M., Karousou, E., De Luca, G. & Passi, A. Metabolic control of hyaluronan synthases. Matrix Biology 35, 8-13, (2014).
30 Zhao, Y. et al. Evolution of high-molecular-mass hyaluronic acid is associated with subterranean lifestyle. Nature Commun 14, (2023).
31 Purushotham, P., Ho, R. & Zimmer, J. Architecture of a catalytically active homotrimeric plant cellulose synthase complex. Science 369, 1089-1094, (2020).
32 Caffalette, C., Corey, R., Sansom, M. S. P., Stansfeld, P. J., Zimmer, J. A lipid gating mechanism for the O antigen ABC transporter. Nature Commun. 10, (2019).
33 Knott, B. C., Crowley, M. F., Himmel, M. E., Zimmer, J. & Beckham, G. T. Simulations of cellulose translocation in the bacterial cellulose synthase suggest a regulatory mechanism for the dimeric structure of cellulose. Chem Sci 7, 3108-3116, (2016).
34 McManus, J., Yang, H., Wilson, L., Kubicki, J. & Tien, M. Initiation, Elongation, and Termination of Bacterial Cellulose Synthesis. ACS Omega 3, 2690-2698, (2018).
35 Liu, H. & Naismith, J. H. An efficient one-step site-directed deletion, insertion, single and multiple-site plasmid mutagenesis protocol. BMC Biotechnol 8, 91, (2008).
36 Dickinson, D. J., Ward, J. D., Reiner, D. J. & Goldstein, B. Engineering the Caenorhabditis elegans genome using Cas9-triggered homologous recombination. Nat Methods 10, 1028-1034, (2013).
37 Studier, F. Protein production by auto-induction in high density shaking cultures. Protein Expr Purif 41, 207-234, (2005).
38 Hilton, M. A. et al. Single-molecule investigations of single-chain cellulose biosynthesis. Proc Natl Acad Sci USA 119, e2122770119, (2022).
39 Errasti-Murugarren, E., Bartoccioni, P. & Palacin, M. Membrane Protein Stabilization Strategies for Structural and Functional Studies. Membranes 11, (2021).
40 Fellouse, F. A. et al. High-throughput generation of synthetic antibodies from highly functional minimalist phage-displayed libraries. J Mol Biol 373, 924-940, (2007).
41 Kim, J. et al. Structure and drug resistance of the Plasmodium falciparum transporter PfCRT. Nature 576, 315-320, (2019).
42 Miller, K. R. et al. T cell receptor-like recognition of tumor in vivo by synthetic antibody fragment. PLoS One 7, e43746, (2012).
43 Jedrzejas, M. J., Mewbourne, R. B., Chantalat, L. & McPherson, D. T. Expression and purification of Streptococcus pneumoniae hyaluronate lyase from Escherichia coli. Protein Expr Purif 13, 83-89, (1998).
44 Punjani, A., Rubinstein, J. L., Fleet, D. J. & Brubaker, M. A. cryoSPARC: algorithms for rapid unsupervised cryo-EM structure determination. Nat Methods 14, 290-296, (2017).
45 Jumper, J. et al. Highly accurate protein structure prediction with AlphaFold. Nature 596, 583-589, (2021).
46 Pettersen, E. F. et al. UCSF Chimera—a visualization system for exploratory research and analysis. J Comput Chem 25, 1605-1612, (2004).
47 Emsley, P. & Cowtan, K. Coot: model-building tools for molecular graphics. Acta Crystallogr D Biol Crystallogr 60, 2126-2132, (2004).
48 Adams, P. et al. PHENIX: a comprehensive Python-based system for macromolecular structure solution. Acta Crystallogr D Biol Crystallogr 66, 213-221, (2010).
49 Pymol. Vol. Version 2.0 (Schroedinger, LLC).
50 Pettersen, E. F. et al. UCSF ChimeraX: Structure visualization for researchers, educators, and developers. Protein Sci 30, 70-82, (2021).
51 Jurrus, E. et al. Improvements to the APBS biomolecular solvation software suite. Protein Sci 27, 112-128, (2018).
52 Edgar, R. C. MUSCLE: multiple sequence alignment with high accuracy and high throughput. Nucl Acid Res 32, 1792-1797, (2004).

All publications, patents, and patent applications, Genbank sequences, websites and other published materials referred to throughout the disclosure herein are herein incorporated by reference to the same extent as if each individual publication, patent, or patent application, Genbank sequences, websites and other published materials was specifically and individually indicated to be incorporated by reference. In the event that the definition of a term incorporated by reference conflicts with a term defined herein, this specification shall control.

1. A method to synthesize Hyaluronan (HA) and modulate HA's length, alter catalytic rate of HA production and/or alter polydispersity of HA comprising expressing a mutant nucleic acid sequence coding for mutant HA synthase (HAS) in a host cell, wherein the mutant HA synthase has one or more substitutions in WGTSGRK/R motif or WGTRG motif.

2. The method of claim 1, wherein the catalytic rate of the mutant synthase is increased or reduced as compared to a wild type synthase.

3. The method of claim 1, wherein the length of HA is shorter or longer compared to HA synthesized with a wild type synthase.

4. The method of claim 1, wherein the polydispersity of the HA is greater or less than HA synthesized with a wild type synthase.

5. The method of claim 1, wherein one or more the amino acids of the WGTSGRK/R motif or the WGTRG motif are individually substituted with any other amino acid.

6. The method of claim 1, wherein Trp of the WGTSGRK/R motif is substituted.

7. The method of claim 5, wherein the Trp is substituted with Ala or Phe.

8. The method of claim 1, wherein Thr of the WGTSGRK/R motif is substituted.

9. The method of claim 8, wherein the Thr is substituted with Ala or Ser.

10. The method of claim 1, wherein Arg of the WGTSGRK/R motif is substituted.

11. The method of claim 10, wherein the Arg is substituted with Ala or Lys.

12. The method of claim 1, wherein at least one Gly in WGTRG is substituted.

13. The method of claim 12, wherein the at least one Gly is substituted with Ala.

14. The method of claim 1, wherein Arg in WGTRG is substituted.

15. The method of claim 14, wherein the Arg is substituted with Lys.

16. A method to synthesize Hyaluronan (HA) and modulate HA's length, alter catalytic rate of HA production and/or alter polydispersity of HA comprising expressing a mutant nucleic acid sequence coding for mutant HA synthase (HAS) in a host cell, wherein the mutant HA synthase has one or more substitutions in a transmembrane (TM) channel.

17. The method of claim 16, wherein the one or more substitutions in the TM channel are at position Arg287, Arg296, H72, and/or K448.

18. The method of claim 17, wherein the one or more substitutions are Arg287Ala, Arg287Lys, Arg296Ala, Arg296Lys, H72A, K448A, K448R, and/or H72F.

19. A method to synthesize Hyaluronan (HA) and modulate HA's length, alter catalytic rate of HA production and/or alter polydispersity of HA comprising expressing a mutant nucleic acid sequence coding for mutant HA synthase (HAS) in a host cell, wherein the mutant HA synthase has one or more substitutions in the active site channel, wherein the substitutions are at C307 or C337.

20. The method of claim 19, wherein C307 and/or C337 are substituted independently with Ala or Ser.

21.-25. (canceled)
